(19) United States
(12) Patent Application Publication
(10) Pub. No.: US 2023/0348892 A1
(43) Pub. Date: Nov. 2, 2023

(54) ENGINEERED MAMALIAN GENETIC CIRCUITS AND METHODS OF USING THE SAME

(71) Applicant: Northwestern University, Evanston, IL (US)

(72) Inventors: Joshua N. Leonard, Evanston, IL (US); Joseph Jacob Muldoon, Evanston, IL (US); Patrick Sean Donahue, Evanston, IL (US)

(73) Assignee: Northwestern University, Evanston, IL (US)

(21) Appl. No.: 18/026,734

(22) PCT Filed: Sep. 16, 2021
(86) PCT No.: PCT/US2021/050584

§ 371 Date: Mar. 16, 2023

Related U.S. Application Data
(60) Provisional application No. 63/079,882, filed on Sep. 17, 2020.

(51) Int. Cl. C12N 15/10

(52) U.S. Cl. CPC C12N 15/1055; C12N 15/1086

(57) ABSTRACT

The present disclosure relates generally to genetic engineering of cells to perform specific and complex functions. In particular, the present disclosure relates to engineered mammalian cells and methods of engineering mammalian cells, as well as novel multi-functional proteins integrating both transcriptional and post-translational control effectively linking genetic circuits with sensors for multi-input evaluations.

### CROSS REFERENCE STATEMENT

This application claims priority under 35 U.S.C. § 119(e) to U.S. Provisional Patent Application No. 63/079,882, filed Sep. 17, 2020, the entire contents of which are incorporated herein by reference.

### GOVERNMENT SUPPORT

This invention was made with government support under grant number EB026510 awarded by the National Institutes of Health (NIH). The government has certain rights in the invention.

### FIELD OF INVENTION

The present disclosure relates generally to genetic engineering of cells to perform specific and complex functions. In particular, the present disclosure relates to engineered mammalian cells and methods of engineering mammalian cells, as well as novel multi-functional proteins integrating both transcriptional and post-translational control effectively linking genetic circuits with sensors for multi-input evaluations.

### BACKGROUND

The following discussion is merely provided to aid the reader in understanding the disclosure and is not admitted to describe or constitute prior art thereto.

Early demonstrations of genetically engineering customized functions in mammalian cells indicate a vast potential to benefit applications including directed stem cell differentiation (1, 2) and cancer immunotherapy (3). In general, most applications require precise control of gene expression and the capability to sense and respond to external cues (4-8). Despite the growing availability of biological parts (such as libraries of promoters and regulatory proteins) that could be used to control cell states, assembling parts to compose customized genetic programs that function as intended remains a challenge, and it often requires iterative experimental tuning or down-selection to identify functional configurations. This highly empirical process limits both the scope of programs that one can feasibly compose and fine-tune and likely the performance of functional programs identified in this manner. Thus, the need for systematic and precise design processes represents a grand challenge in the field of mammalian synthetic biology.

Model-guided predictive design has been demonstrated in the composition of some cellular functions, including transcriptional logic in bacteria (9) as well as logical (10) and analog behaviors in yeast (11); however, this type of approach is less developed in mammalian systems. To date, transcription factors (TFs) based on zinc fingers (ZFs) (12, 13), transcription activator-like effectors (TALEs) (14-17), dCas9 (18, 19), and other proteins (20) have been used to implement transcriptional logic in mammalian cells. Some of these studies make use of protein splicing (12, 14, 18). Other studies have used RNA-binding proteins (21), proteases (22, 23), and synthetic protein-binding domains (17). Yet, none of these approaches currently enable the customized design of sophisticated mammalian cellular functions and prediction of circuit performance based only upon descriptions of the component parts. Associated challenges include the availability of appropriate parts (24), suitably descriptive models that support predictions using these parts (25), and computational and conceptual tools that facilitate the identification of designs that function robustly despite biological variability and crosstalk (26-28).

Accordingly, there is a need in the art for a tractable set of genetic circuits useful in mammalian cells that do not require unduly laborious testing and empirical trial-and-error tuning. The present disclosure fulfills that need by providing such genetic circuits with a variety of functions including, but not limited to, digital and analog information processing, and sense-and-respond behaviors.

### SUMMARY

Genetically engineering cells to perform customizable functions is an emerging frontier with numerous technological and translational applications. However, it remains challenging to systematically engineer mammalian cells to execute complex functions. To address this need, the present disclosure provides method enabling accurate genetic program design using high-performing genetic parts and predictive computational models, as well as novel multi-functional proteins integrating both transcriptional and post-translational control, validated models for describing these mechanisms, implemented digital and analog processing, and effectively linked genetic circuits with sensors for multi-input evaluations. The functional modularity and compositional versatility of these parts enable one to satisfy a given design objective via multiple synonymous programs. The platform described herein enables bioengineers to predictively design mammalian cellular functions that perform as expected even at high levels of biological complexity.

In one aspect, the disclosure provides an engineered genetic circuit comprising: (a) one or more engineered proteins selected from the group consisting of: an engineered protein that activates gene expression, wherein the engineered protein comprises a DNA binding domain, a transcription activator domain, and at least one split intein on the C-terminus or N-terminus of the DNA binding domain and/or the transcription activator domain; an engineered protein that inhibits gene expression, the engineered protein comprising a DNA binding domain, a transcription inhibitor domain, and at least one split intein on the C-terminus or N-terminus of the DNA binding domain and/or the transcription inhibitor domain; and a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind, and wherein and the first engineered protein and the second engineered protein each comprise at least one split intein; and (b) one or more engineered expression vectors comprising a minimal promoter and one or more DNA binding sites for the DNA binding domains of the engineered proteins of (a), and optionally a gene of interest that is expressed from the minimal promoter.

In some embodiments, the genetic circuit may comprise the engineered protein of (i) and the engineered protein of (ii).

In some embodiments, the DNA binding domain of the one or more engineered proteins of (i), (ii), and (iii) comprises one or more zinc fingers. In some embodiments, the zinc finger may be such as ZF1, ZF2, ZF3, ZF4, ZF5, ZF6, ZF7, ZF8, ZF9, ZF10, ZF11, ZF12, ZF13, ZF14, or ZF15. In some embodiments, the DNA binding domain of the one or more engineered proteins of (i), (ii), and (iii) may comprise, for example, all of or a functional fragment of a zinc finger protein comprising more than three DNA-binding domains, other classes of programmable DNA binding domains (e.g., transcription activator-like effector (TALE)), DNA binding domains derived from microbial proteins (e.g., tetR, 1acI, etc.), and/or Cas9 or variants of Cas9 and other Cas proteins, including catalytically inactive variants (e.g., dCas9). In some embodiments, the DNA binding domain of the one or more engineered proteins of (i), (ii), and (iii) comprises 2, 3, or more zinc fingers or 2, 3, or more of the other DNA binding domains provided herein.

In some embodiments, the engineered proteins are fusion proteins comprising heterologous domains.

In some embodiments, the transcription activator domain of the engineered protein of (i), (ii), and/or (iii) comprises a domain from a transcription activator selected from the group consisting of Herpes simplex virus protein 16 (VP16), a synthetic tetramer of VP16 (VP64), nuclear factor (NF) kappa-B (p65), heat shock transcription factor 1 (HSF1), replication and transcription activator (RTA) of the gamma-herpesvirus family, p53, an acidic domain (also known as “acid blobs” or “negative noodles,” rich in D and E amino acids, present in Ga14, Gcn4 and VP16), a glutamine-rich domain (which may comprise multiple repetitions like “QQQXXXQQQ,” like those present in transcription factor Sp1), a proline-rich domains (which may comprise repetitions like “PPPXXXPPP,” like those present in c-jun, AP2, and October 2), an isoleucine-rich domain (which may comprise repetitions of “IIXXII,” like those present in NTF-1), and a multipartite activator.

In some embodiments, the engineered protein of (ii) inhibits activation of transcription by the engineered protein of (i).

In some embodiments, the transcription regulator domain of the second engineered protein of the combination of engineered proteins of (iii) is a transcription activator domain optionally selected from the group consisting of Herpes simplex virus protein 16 (VP16), a synthetic tetramer of VP16 (VP64), nuclear factor (NF) kappa-B (p65), heat shock transcription factor 1 (HSF1), replication and transcription activator (RTA) of the gamma-herpesvirus family, p53, an acidic domain (also known as “acid blobs” or “negative noodles,” rich in D and E amino acids, present in Ga14, Gcn4 and VP16), a glutamine-rich domain (which may comprise multiple repetitions like “QQQXXXQQQ,” like those present in transcription factor Sp1), a proline-rich domains (which may comprise repetitions like “PPPXXXPPP,” like those present in c-jun, AP2, and October 2), an isoleucine-rich domain (which may comprise repetitions of “IIXXII,” like those present in NTF-1), and a multipartite activator.

In some embodiments, the engineered proteins of (i) or (ii) are present in an exogenous extracellular sensor. In some embodiments, the extracellular sensor comprises: (a) a ligand binding domain, (b) a transmembrane domain, (c) a protease cleavage site, and (d) the engineered protein of (i) or (ii).

In some embodiments, the split intein is a wild-type split intein. In some embodiments, the at least one split intein is a mutated split intein. In some embodiments, the at least one split intein is appended to the N-terminus of the engineered protein. In some embodiments, the split intein comprises SEQ ID NO: 1 or an amino acid sequence that possesses at least about 70%, at least about 75%, at least about 80%, at least about 85%, at least about 90%, at least about 95%, at least about 96%, at least about 97%, at least about 98%, or at least about 99% identity thereto. In some embodiments, the at least one split intein is appended to the C-terminus of the engineered protein. In some embodiments, the split intein comprises SEQ ID NO: 3 or an amino acid sequence that possesses at least about 70%, at least about 75%, at least about 80%, at least about 85%, at least about 90%, at least about 95%, at least about 96%, at least about 97%, at least about 98%, or at least about 99% identity thereto.

In some embodiments, the circuit components are eukaryotic, while in some embodiments, the circuit components are mammalian.

In some embodiments, the stimulus is a ligand, exposure to light, removal from light, phosphorylation, dephosphorylation, a post-translational modification of the dimerization domain, a change in the state of the environment in which the engineered genetic circuit is expressed.

In another aspect, the present disclosure provides an engineered genetic circuit, comprising: (a) a first engineered protein that activates gene expression, the first engineered protein comprising a first DNA binding domain, a first transcription activator domain, and at least one split intein on the C-terminus or N-terminus of the first DNA binding domain and/or the first transcription activator domain; (b) a first engineered expression vector comprising a minimal promoter and first DNA binding sites for the first DNA binding domain of the first engineered protein, and a first gene of interest that is expressed from the minimal promoter, wherein the gene of interest encodes a second engineered protein, the second engineered protein comprising a second DNA binding domain, a second transcription activator domain, and at least one split intein on the C-terminus or N-terminus of the second DNA binding domain and/or the second transcription activator domain; and (c) a second engineered expression vector comprising a minimal promoter and second DNA binding sites for the second DNA binding domain of the second engineered protein, and a second gene of interest that is expressed from the minimal promoter, wherein the second gene of interest encodes a detectable reporter protein; wherein the first engineered protein increases expression from the first engineered expression vector and the second engineered protein increases expression from the second engineered vector.

In another aspect, the present disclosure provides an exogenous extracellular sensor system comprising: (i) a first exogenous extracellular sensor component comprising:
(a) a ligand binding domain,
(b) a transmembrane domain,
(c) a protease cleavage site, and
(d) an engineered protein domain comprising a DNA binding domain, a transcription activator domain, and at least one split intein on the C-terminus or N-terminus of the DNA binding domain and/or the transcription activator domain;

(ii) a second exogenous extracellular sensor component comprising
(a) a ligand binding domain,
(b) a transmembrane domain, and
(c) a protease domain; and, optionally,

(iii) an engineered expression vector comprising a minimal promoter and one or more DNA binding sites for the DNA binding domains of the first exogenous extracellular sensor, and, optionally, a gene of interest that is expressed from the minimal promoter; wherein the ligand binding domain of the first exogenous extracellular sensor component and the ligand binding domain of the second exogenous extracellular sensor component bind to the same ligand to form a tertiary complex; wherein the protease domain of the second exogenous extracellular sensor component cleaves the protease cleavage site of the first exogenous extracellular sensor component to release the engineered protein domain comprising the DNA binding domain and transcription activator domain; and wherein the DNA binding domain of the engineered protein domain binds to the one or more DNA binding sites of the engineered expression vector and increases expression from the minimal promoter of the engineered expression vector.

In another aspect, the present disclosure provides a host cell comprising the engineered genetic circuit of any one of foregoing aspects or embodiments or the exogenous extracellular sensor system of the foregoing aspect. In some embodiments, the host cell is eukaryotic. In some embodiments, the host the cell is mammalian.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIG. 1 shows logical evaluation is enabled by transcriptional and post-translational regulation. (A,B) Cartoons depict (A) the genetic components and (B) their arrangement and use in simulations to produce intended functions. Transcription is mediated by COMET TFs, which here are modified with split inteins to incorporate post-translational regulation via splicing. Genetic parts that carry out specified activities and that can be described mathematically enable the predictive customization of cellular functions. In the schematics, circles are protein domains, arrows indicate splicing or regulation, yellow highlighting denotes the inputs, and the red node is the output. (C-J) A panel of logic gates was designed, simulated, and experimentally evaluated. Synthetic digital logic in cells is inherently analog, and component doses were selected to examine this behavior and underscore particular features (e.g., in C, reporter signal decreases at a high intC-ZF1 dose because intC-ZF1 inhibits ZFa-mediated transcription). In the electronic diagrams (teal background), lines denote splicing or regulation. Processes that have a modest effect within the dose range examined, and that because of fundamentally analog behavior do not carry out a fully digital function, are denoted by dotted lines. In the mechanistic diagrams (blue background), purple bent arrows are promoters, and black arrows indicate splicing and regulation. Yellow highlighting denotes the components for which dose is varied (in gene copies). Simulation and experimental results are presented in heatmaps that indicate how the two inputs affect reporter output (mKate2 signal in MEPTRs); color-coding denotes the mean reporter signal from three biological replicates (bar graphs in FIG. 15, histograms in FIG. 16), scaled by the maximum value in each heatmap. Simulations in C are from a fit to the data, and subsequent panels (D-J) are predictions. (K) Some of the motifs that were used in the gate designs confer sharp transitions in reporter output. For example, a standard activation dose response was not ultrasensitive, but layering two inhibitors in a cascade did produce ultrasensitivity (Hill coefficient n>1). The downstream inhibitor is tagged with a PEST degron.

FIG. 2 shows compact multi-output logic is attained through functional modularity. (A) A strategy for multi-output logic is proposed by using multi-tasking proteins that retain the functions of their constituent domains. The cartoons depict the use of multiple DNA-binding domains on a TF to regulate multiple genes, the embedding of a split intein fragment within a functioning TF to enzymatically alter its activity, and the merging of features from multiple genetic programs to enable their compact simultaneous implementation. (B-F) A panel of multi-input-multi-output gates was designed, simulated, and experimentally evaluated. As an example, C is deconstructed to show how separate topologies containing proteins that have some domains in common and are amenable to the appending of additional domains can be compressed. In the plots, color-coding denotes the mean mKate2 and EYFP reporter signal from three biological replicates (bar graphs in FIG. 23), scaled by the maximum value in each heatmap. (G) These plots summarize the complexity of the gates that were designed and validated in FIG. 1 (red) and FIG. 2 (purple), with complexity defined based on the size and depth of the circuits in the electronic diagrams (upper) or based on the numbers of genes, regulatory connections, and regulatory proteins employed (lower). The expanded toolkit of genetic parts and model-guided approach were successful for building circuits spanning a range of attributes, which suggests that this design process could be executed reliably for many future objectives.

FIG. 3 shows analog behaviors are constructed by using TFs that play multiple roles. Reconstitutable TFs have dose response properties that are conducive to analog signal processing. Simulated and experimentally observed responses are shown relating to (A-C) ultrasensitivity and (D-I) bandpass concentration filtering. Several designs were evaluated for the ability to meet these objectives. To implement ultrasensitivity, the Hill coefficient (n) was most effectively increased through a strategy of removing an inhibitor in the process of producing an activator (C). To implement bandpass concentration filtering, a tighter upper threshold was best achieved through a similar strategy that also included additional regulation: moderate levels of FKBP-ZF act primarily to reconstitute RaZFa, and high levels of FKBP-ZF act to inhibit the reporter and VP16-FRB (G). Simulations in A and D are fitted to data, and the other panels are predictions. The prediction plots present simulations for how output gene expression varies with dose of the component highlighted in yellow; each plot includes a set of responses varying the component highlighted in red-to-blue gradation. Doses for the x-axes and above the varied component in the diagrams are in plasmid ng. Each experimental plot corresponds to the simulated condition with the dark line (for the middle dose of the varied component). The ZF1/2x6-C promoter has six partially overlapping ZF1 and ZF2 sites. DMSO is the vehicle for rapamycin, which is used here as an environmental species (not an input). The simulations with RaZFa correspond to conditions with rapamycin treatment. Experiment plots represent the mean and S.E.M. of EYFP reporter signal from three biological replicates (bar graphs in FIG. 26).

FIG. 4 shows sensors can be linked to genetic programs to make signaling cascades. MESA and COMET technologies can be combined to construct functional biosensors, and upstream biosensor output is well-matched to the requirements for downstream promoter input. (A,B) ABA-ZF2a and Rapa-MESA-ZF6a each exhibit ligand-inducible signaling (p=2×10⁻³ and p=1×10⁻³, respectively, one-tailed Welch's unpaired t-test). EtOH is the vehicle for both ligands. For MESA, the TC contains an FRB ectodomain and intracellular COMET TF, and the PC contains an FKBP ectodomain and intracellular TEV protease (TEVp). Each receptor chain contains an FGFR4 transmembrane domain. (C-E) Validated sensors were applied to implement multi-input sensing. AND logic was selected as a design goal, and four synonymous topologies—those that are intended to achieve the same goal through different mechanisms—were proposed and evaluated. For each input type (two columns for upstream ZFa or ligand sensing) and topology (four rows), reporter signal with two inputs differed from that with either or no input (p<2×10⁻¹⁶ in each case, three-factor ANOVA and Tukey's HSD test), indicating successful AND gate outcomes. Topologies 2-4 displayed negligible background signal (comparable to the signal with only the reporter present, ˜10¹-10² MEPTRs, FIG. 33), despite involving multi-layer signaling which can be a potential source of leak. The (ZF2/ZF6)×3 promoter has three pairs of alternating ZF2 and ZF6 sites. Bar graphs represent the mean, S.E.M., and values of mKate2 reporter signal from three biological replicates (depicted as dots; near-zero values are below the log-scaled y-axis lower limit). The numbers above bar pairs are the fold difference, and a fold difference of ∞ indicates that the denominator signal is less than or equal to zero.

FIG. 5 shows development of the genetic components and models. Flow cytometry gating was used to identify live cells (FSC-A vs. SSC-A), singlet events (FSC-A vs. FSC-H), and EBFP2 (transfection control) expression (Pacific Blue channel). The EBFP2+ gate was defined by setting a threshold to include the top ˜1% of live single-cell events for cells transfected with empty vector only.

FIG. 6 shows trends in mKate2 reporter signal for ZF1-based activator dose responses vary depending on choice of constitutive promoter (CMV, EF1α) and activation domain (VP16, VP64); the combinations are color-coded. These are the types of dose responses from which transcriptional activation parameters can be estimated (Example 1—Materials and Methods). Component doses indicate the amount transfected per well in units of either gene copies or plasmid mass. Diagrams use these conventions: purple right-angle arrows are promoters; black arrows are transitions (splicing, ligand binding, proteolytic cleavage) or transcriptional regulation (activation, inhibition); highlighting is for components for which dose is varied. In these diagrams, RNA species and the transfection control marker EBFP2 are not depicted.

FIG. 7 shows plots that display cross-sections of the data for EYFP reporter signal across VP16-ZF1 activator plasmid doses and EYFP reporter plasmid doses, which are color-coded. Reporter signal follows an approximately square root relationship with reporter plasmid dose, and this trend holds across activator plasmid doses; this observation was used to arrive at the heuristic in Eqn. 4 (see computational methods subsection of Example 1—Materials and Methods).

FIG. 8 shows COMET TF function can be reconstituted using gp41-1 split inteins. The split inteins comprise an N-terminal fragment (intN) and a C-terminal fragment (intC) that can be used to ligate proteins. The splicing reaction proceeds as A-intN-B+X-intC-Y →A-Y+X-intC/intN-B, where A, B, X, and Y denote adjoining protein sequences (exteins) or a lack thereof, “-” denotes a peptide bond, and “/” denotes a non-covalent interaction. Here, activation is reconstituted by ligating ZF1 and VP64. The results indicate that in the absence of intC on ZF1, co-expression of VP64-intN and ZF1 does not induce reporter expression. For the histograms, y-axes are scaled to distinguish each condition, such that the near-zero-signal region of each histogram is truncated. Color-coding indicates intC-ZF1 and ZF1 gene copies.

FIG. 9 shows Model parameters for split intein-mediated reconstitution (rec) and degradation of intC-containing proteins (k_{degintC}) were fit using the AND gate data in FIG. 1C. The diagram depicts the state variables including RNAs and proteins. Color-coding indicates VP64-intN gene copies.

FIG. 10 shows a statistical model for observed variation in gene expression was used to simulate heterogeneous cell populations (Example 1—Materials and Methods). This approach was first developed in (73) and later used in (60). The flow cytometric distributions (collected in the same experiment) are EBFP2 signal and EYFP signal after gating on EBFP2-expressing (transfected) cells. The current study uses a statistical model based on the distribution observed when cells are harvested for flow cytometry using a trypsin digest; this model differs from those previously utilized in a study in which cells were harvested by a slightly different but common method (FACS buffer harvest) (60). A key lesson is that the method used to prepare cells for flow cytometry affects the observed fluorescence distribution, and thus the mathematical description should be matched to the choice of experimental method. An experiment like the one in this figure could be employed to generate a statistical distribution for any given method of gene delivery and cell harvest as needed. The right panels are unit-normalized simulated distributions of gene expression and simulated correlations for co-expressed genes on transfected plasmids, all based on the generated population Z matrix. This matrix is used to introduce heterogeneity into dynamical models by applying unitless multipliers to the rates of transcription from each plasmid (columns) from each cell (rows). The algorithm for generating Z as described in (60) ensures that relative expression from each plasmid species is similarly distributed across cells (upper right) and that expression across plasmid species is similarly correlated (r˜0.8) within cells (lower right).

FIG. 11 shows the incorporation of split inteins provides new ways to utilize TFs. Homogeneous (single-cell) simulations were conducted for a panel of proposed motifs to survey the responses that could be generated beyond COMET base cases (i, ii). Plots below each diagram display cross-sections of plasmid dose responses: one component is varied along the x-axis, and the other component is varied as specified by the blue color-coding. The descriptor above each plot indicates whether the dose response outcome is activating or inhibitory, or if these outcomes vary depending on the dose of the other component. The heatmaps provide more continuous depictions of outcomes (red color-coding scaled to the maximum value across the ten cases), and the descriptor above each heatmap indicates the type of gate that each outcome resembles. The new motifs (iii-x) broaden the types of responses that can be generated.

FIG. 12 shows that since the emission spectrum of mKate2 overlaps with that of DsRed-Express2 (abbreviated as DsRed), a modification was made to remove this fluorescent signal was from the DsRed-ZF inhibitor. An R95K mutation (74) was introduced to the catalytic triad of the chromophore to generate a variant that was termed DsDed-ZF. This TF produced no detectable DsRed signal, and it retained inhibitory potency against a VP64-ZF1 activator as evident from the low EYFP reporter signal.

FIG. 13 shows a panel of splicing-based circuits was proposed, and population simulations were conducted to identify designs for experimental implementation and testing. The descriptor above each diagram indicates the gate(s) that each outcome matches and/or resembles (where “-like” indicates imperfect performance for the stated gate outcome). In (v-xvi), the plasmid dose of constitutive VP64-ZF1 was set to 1 ng. Circuits diagrammed with a dark background (i, vi, x, xviii) were among those tested in FIG. 1.

FIG. 14 shows a comparison of reporter signal in standard activation and in a double inversion cascade. The cascade produces an ultrasensitive response; n is the exponent from the Hill equation (Eqn. 28; see computational methods subsection of Example 1Materials and Methods) fitted to data. For the histograms, y-axes are scaled to distinguish each condition, such that the region near zero signal of each histogram is truncated. Color-coding indicates component doses.

FIG. 15 shows bar graphs representing the mean and S.E.M. of reporter signal from three biological replicates. Data correspond to FIG. 1C-J. Data for (i, ii vii), (iii, iv, v, viii), and (vi) were collected separately.

FIG. 16 shows data and simulations corresponding to FIG. 1C-J as representative flow cytometry histograms.

FIG. 17 shows data and simulations corresponding to FIG. 1C-J as (left column) goodness of prediction (Q²), Spearman's rank correlation coefficient squared (p²), and mean squared error (MSE) comparing max.-normalized simulated and observed mean signal; and as (right column) difference heatmaps for max.-normalized simulated and observed mean signal. The Q² values indicate that for all eight gates, the simulations explain the majority of the variance in the data; five gates provide very close agreement (>90% Q²).

FIG. 18 shows MIMO gate designs and single-cell outcomes for NIMPLY/NOT corresponding to FIG. 2B. These data were collected in the same experiment as the data for FIG. 2. Compression of genetic programs to arrive at MIMO designs, population simulations (200 cells; signals in model-specific a.u.), and corresponding representative flow cytometric distributions (in MFI) for mKate2 and EYFP reporter signals. This task distribution is also evident in the experimental data. This result highlights how the approach used here for representing cell heterogeneity can capture different types of population outcomes. All of the distribution plots use logicle axis scaling. Orange outlines for three of the gates denote cases in which some cells are predicted to substantially express at most one reporter or the other but not both.

FIG. 19 shows MIMO gate designs and single-cell outcomes for IF/NIMPLY corresponding to FIG. 2C. These data were collected in the same experiment as the data for FIG. 2. Compression of genetic programs to arrive at MIMO designs, population simulations (200 cells; signals in model-specific a.u.), and corresponding representative flow cytometric distributions (in MFI) for mKate2 and EYFP reporter signals. This task distribution is also evident in the experimental data. This result highlights how the approach used here for representing cell heterogeneity can capture different types of population outcomes. All of the distribution plots use logicle axis scaling.

FIG. 20 shows MIMO gate designs and single-cell outcomes for IF/AND corresponding to FIG. 2D. These data were collected in the same experiment as the data for FIG. 2. Compression of genetic programs to arrive at MIMO designs, population simulations (200 cells; signals in model-specific a.u.), and corresponding representative flow cytometric distributions (in MFI) for mKate2 and EYFP reporter signals. This task distribution is also evident in the experimental data. This result highlights how the approach used here for representing cell heterogeneity can capture different types of population outcomes. All of the distribution plots use logicle axis scaling.

FIG. 21 shows MIMO gate designs and single-cell outcomes for NIMPLY/AND corresponding to FIG. 2E. These data were collected in the same experiment as the data for FIG. 2. Compression of genetic programs to arrive at MIMO designs, population simulations (200 cells; signals in model-specific a.u.), and corresponding representative flow cytometric distributions (in MFI) for mKate2 and EYFP reporter signals. This task distribution is also evident in the experimental data. This result highlights how the approach used here for representing cell heterogeneity can capture different types of population outcomes. All of the distribution plots use logicle axis scaling. Orange outlines for three of the gates denote cases in which some cells are predicted to substantially express at most one reporter or the other but not both.

FIG. 22 shows MIMO gate designs and single-cell outcomes for NIMPLY/NIMPLY corresponding to FIG. 2F. These data were collected in the same experiment as the data for FIG. 2. Compression of genetic programs to arrive at MIMO designs, population simulations (200 cells; signals in model-specific a.u.), and corresponding representative flow cytometric distributions (in MFI) for mKate2 and EYFP reporter signals. This task distribution is also evident in the experimental data. This result highlights how the approach used here for representing cell heterogeneity can capture different types of population outcomes. All of the distribution plots use logicle axis scaling. Orange outlines for three of the gates denote cases in which some cells are predicted to substantially express at most one reporter or the other but not both.

FIG. 23 shows bar graphs representing the mean and S.E.M. of reporter signal corresponding to FIG. 2B-F for three biological replicates.

FIG. 24 shows Q², p², and MSE comparing max-normalized simulated and observed signals corresponding to FIG. 2B-F. The comparisons indicate that in most cases, model simulations can explain the majority of the variance in the experimental data, and several cases have very close agreement (≥90% Q²).

FIG. 25 shows analog signal processing. (A-C) Split intein-based programs were not substantially more ultrasensitive than a standard activation dose response; n is the exponent from the Hill equation fitted to the monotonically increasing portion of the data. Despite an apparent similarity of the split intein design in Panel B and the RaZFa design in FIG. 3C, these systems have different ultrasensitivity outcomes. This difference is due at least in part to decreased protein stability conferred by the intC domain, in that there would be less unspliced intC-ZF available than there would be non-dimerized FKBP-ZF for the same DNA dose, and thus less protein to act as an inhibitor in the former case. Data were collected in the same experiment. The mean and S.E.M. are plotted both as dot plots and bar graphs.

FIG. 26 shows data corresponding to FIG. 3. Specifically, panels A-I of this figure correspond to panels A-I of FIG. 3. Comparisons are of the max.-normalized simulated and observed mean reporter signal. The comparisons indicate that in each case, model simulations can explain the majority of the variance in the experimental data, and some cases have very close agreement (>90% Q²).

FIG. 27 shows population simulations for hypothetical variations on the topology investigated in FIG. 3G. Three regulatory connections—(1) FKBP-ZF-mediated inhibition of VP16-FRB expression, (2) RaZFa-mediated induction of VP16-FRB expression, and (3) FKBP-ZF-mediated inhibition of reporter expression—are included or withheld in eight combinations. The lower-right scenario is the true scenario (the case which was experimentally evaluated in FIG. 3G) with all three connections included. The simulations indicate scenarios that are expected to produce bandpass filtering. Tight filtering is most evident in scenarios (vi) and (viii), which suggests that connections 1 and 3 contribute more than does 2 towards this design goal.

FIG. 28 shows receptor engineering and integration of ligand-responsive components. Homogeneous (single-cell) simulations suggest that different numbers of COMET transcriptional layers lead to altered dose responses (varied steepness and plateau of reporter expression) but do not produce a substantial leak in reporter signal. This absence of background signal propagation would support the implementation of high-performance multi-layer cascades. ZFa activities at target promoters are treated as orthogonal, and for simplicity all layers are assigned the same parameters using the base case of VP64-ZF1 at a ZF1x6-C promoter. Plasmid doses: ZFa #1 varied along the x-axis, ZFa #2 varied by color-coding, and ZFa #3 varied across plots.

FIG. 29 shows a strategy that was investigated for modulating split intein splicing efficiency. 11 glutamine and lysine residues across intN and intC were selected based on crystallographic evidence (58) for electrostatic involvement in the initial capture step in the mechanism for gp41-1 folding and splicing. Mutating residues to alanine (either individually or in combination, to reduce the number of electrostatic interactions) was hypothesized to decrease the likelihood of intN and intC folding upon coming into contact with each other—and by extension decrease TF reconstitution efficiency—and that this effect would be greater for intC as membrane-proximal cargo on a MESA target chain (TC) than as an intracellular protein. A sufficient differential effect between these two contexts would enable effective fusion of intC-ZF1 onto a TC, such that interactions with intracellular intN-containing components would occur only after proteolytic cargo release in ligand-induced receptor signaling. Abbreviations: ectodomain (ECD), transmembrane domain (TMD). (A) The cartoon illustrates the evaluation of reconstitution between two intracellular components (VP64-intN and intC-ZF1) and between an intracellular component and a receptor (VP64-intN and Rapa-MESA TC:intC-ZF1). Mutations were considered ideal if reporter signal was retained in the former scenario and not produced in the latter. (B-C) Several mutants were generated and tested (B), and based on these results, double mutants with K45A for intN and with E104A for intC were generated and tested (C). Heatmaps denote the mean reporter signal from three biological replicates (left heatmaps) and the fold difference in mean signal with intC-ZF1 vs. TC:intC-ZF1 (right heatmaps). The results indicate that the new pairings disrupted interactions with intN more for TC-fused intC than for intracellular intC. In some cases, pairings also produced up to several fold greater reporter signal in the intracellular context than did the WT-WT case, and in tandem with the reduction in signal in the receptor context there was a several thousand-fold context-dependent difference. High-performing variants were carried forward for further investigation (FIG. 30). For the mutation of all 11 residues using the pairing of intN fiveA (K41A, K43A, K45A, K48A, E52A) and intC sixA (K92A, K93A, E98A, E99A, E102A, E104A) (B), reporter expression was not induced in either context. Thus, mutations can be used tune reconstitution efficiency from WT level to effectively none, and there exists an intermediate regime with a differential effect based on whether intC is TC cargo or intracellular.

FIG. 30 shows a functional assay that was conducted for ligand-inducible receptor signaling incorporating split inteins. A panel of intC and intN pairings from the mutagenesis assay was evaluated for compatibility with the MESA signaling mechanism by measuring reporter signal in three scenarios: (1) VP64-intN and TC:intC-ZF1 with vehicle (EtOH), (2) VP64-intN, TC:intC-ZF1, and MESA protease chain (PC) with vehicle, and (3) VP64-intN, TC:intC-ZF1, and PC with receptor ligand (rapalog). Outcomes are considered ideal if reporter signal is low in the first two scenarios and high in the third. However, it was observed that for each pairing, reporter signal was similar regardless of PC co-expression or ligand treatment. This result does not support the ability of PC to cleave intC-containing cargo from the TC, and instead indicates that the conditions in which reporter signal was observed were due to residual interactions between intracellular intN and TC-bound intC partial-mutant variants. These designs were not carried forward, but variants with different intracellular linker lengths were evaluated in the next panel.

FIG. 31 shows a functional assay to assess the effect of TC intracellular linker length. Introducing more physical distance or geometric flexibility between the protease recognition sequence (PRS) and cargo was hypothesized to enable PC-mediated cleavage of TC:intC-ZF1. The base case TC linker had one glycine-serine (GS) repeat, and between one and five repeats were tested for mutant intC-ZF1 cargo co-expressed with mutant VP64-intN and for VP64-ZF1 cargo. For the case with intC-ZF1 cargo, reporter signal increased with increasing linker length; however, a signal increase occurred regardless of PC co-expression and ligand treatment. Therefore, modifications to the TC membrane-proximal region did not alleviate the inability of the PC to cleave these TCs. It is possible that the effect of increasing linker length is to make the membrane-proximal intC more accessible (resembling intracellular intC) to intracellular intN. For the case with VP64-ZF1 cargo, there was low reporter signal with PC and no ligand, and there was high reporter signal with PC and ligand, demonstrating for the first time that a ZFa can be effectively used on MESA. For increasingly long linkers, the reporter signal decreased. Based on these findings, full transcription factors, such as ZFa, were used as cargo. Linkers for subsequent TCs (FIG. 4, FIGS. 32-33) contained one GS repeat.

FIG. 32 shows MESA with DsDed-ZF1 cargo can ligand-inducibly signal to inhibit target gene expression. In this assay, treatment with EtOH or rapalog was applied both at the time of transfection and at the time of media change (rather than only at the latter) to promote inhibitory signaling upon expression of the receptor, analogously to inhibition that could take place upon expression of the intracellular inhibitor. In summary, for FIGS. 29-32, a strategy of mutating charged residues at the intN and intC fragment interface was pursued to counter their association. Functional assays identified mutants that prevented intC-ZF1 cargo from reconstituting with soluble VP64-intN and that did not prevent soluble intC-ZF1 from doing so, suggesting that cargo could be isolated from downstream activity without precluding activity. With certain mutations, it was also possible to tune soluble intC-ZF1 and VP64-intN splicing down to zero (FIG. 29). However, full MESA signaling was not observed with intC cargo (FIGS. 30-31), but since inducible signaling did prove to be effective with ZFa cargo, this configuration was selected.

FIG. 33 shows data corresponding to FIG. 4 with linear y-axis scaling. Split intein-containing components were placed under the control of COMET inducible promoters and incorporated downstream of either constitutive TFs or a ligand-responsive TF and receptor. Data were collected in the same experiment.

FIG. 34 shows selected examples of antagonistic bifunctionality, in which a component exerts opposing effects.

FIG. 35 shows a depiction of validated multi-domain regulators used (or generated through reactions) in this study. Even a relatively small set of domains can be combined in numerous ways, such that multiple activities can be concisely encoded in TFs. An additional note: after small molecule-reconstituted TFs dimerize, the small molecule-binding domains are not involved in subsequent interactions, and because the remaining functional domains (those that can carry out subsequent activities) are the AD and ZF, the small molecule-reconstituted TFs are depicted in the category for two functional domains.

### DETAILED DESCRIPTION

The present disclosure provides a platform of composable mammalian elements of transcription (COMET) with novel functionalities and which may be integrated in novel ways. The present disclosure achieves the incorporation of protein splicing-based post-translational control in COMET transcription factors, which can be accurately predicted using mathematical models. This mechanism employs split inteins: complementary domains that fold and trans-splice to covalently ligate flanking domains. The present disclosure demonstrated the utility of this platform by designing and building genetic circuits that implement a variety of functions including digital and analog information processing and sense-and-respond behaviors. Part of this implementation includes demonstrating the combination of modular expression sensor architecture (MESA) receptors and COMET transcription factors. Ultimately, this capability enables the construction of sophisticated engineered cellular functions for applications in biotechnology, medicine, and fundamental research.

I. Definitions

It is to be understood that methods are not limited to the particular embodiments described, and as such may vary. It is also to be understood that the terminology used herein is for the purpose of describing particular embodiments only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a peptide” includes a single peptide as well as a plurality of peptides, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “about” means plus or minus 10% as well as the specified number. For example, “about 10” should be understood as both “10” and “9-11.”

As used herein, “optional” or “optionally” means that the subsequently described event or circumstance may or may not occur, and that the description includes instances where said event or circumstance occurs and instances where it does not.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding polynucleotide and amino acid sequences, “variant,” “mutant,” or “derivative” may be defined as a sequence having at least 50% sequence identity to the particular sequence over a certain length of one of the sequences using blastn or blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of variant, mutant, or derivative sequences may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode the same or similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

“Transformation” or “transfection” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g. U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide. Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g., the fusion protein disclosed herein).

Certain proteins or polypeptide sequences disclosed herein (e.g., split inteins) may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant”, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

II. Abbreviations

| | |
|---|---|
| ABA | Abscisic acid |
| ABA-ZFa | Abscisic acid-activated zinc finger activator |
| ABI1 | Abl interactor 1 |
| AD | Activation domain |
| CMV | Cytomegalovirus promoter |
| COMET | COmposable Mammalian Elements of Transcription |
| DMSO | Dimethyl sulfoxide vehicle for rapamycin treatment |
| DsDed | R95K mutant for DsRed-Express2 |
| DsRed | Abbreviation used for DsRed-Express2 |
| ECD | Ectodomain |
| EF1α | Elongation factor-1 alpha promoter |
| EtOH | Ethanol vehicle for rapalog treatment and abscisic |
| | acid treatment |
| FKBP | FK506 binding protein |
| FRB | FKBP-rapamycin binding |
| intC | Split intein C-terminal fragment |
| intN | Split intein N-terminal fragment |
| MEFL | Molecules of Equivalent Fluorescein |
| MEPTR | Molecules of Equivalent PE-Texas Red |
| MESA | Modular Extracellular Sensor Architecture |
| MFI | Mean fluorescence intensity |
| MIMO | Multi-input multi-output |
| PC | Protease chain |
| PRS | Protease recognition sequence |
| PYL1 | Pyrabactin resistance 1-like |
| RaZFa | Rapamycin-activated zinc finger activator |
| TC | Target chain |
| TF | Transcription factor |
| TMD | Transmembrane domain |
| VP16 | VP16 activation domain |
| VP64 | VP64 activation domain |
| ZF | Zinc finger |
| ZFa | Zinc finger activator |

III. Predictable Engineered Genetic Circuit

The technical field of the disclosed platform technology relates to biological engineering in mammalian synthetic biology. Mammalian cells can be programmed for numerous applications, ranging from customized cell-based therapeutics to tools for probing fundamental biological questions. To date, however, the tools available for composing such biological programs are limited in number, and tuning the performance of such biological parts is challenging, limiting the scope of applications that can be pursued. To meet this need, a Composable Mammalian Elements of Transcription (COMET) toolkit has been developed, and the current technology platform builds onto the COMET toolkit—making it more precise and, simultaneously, more versatile and genetically compact—by incorporating into the genetic circuits split parts (e.g., by utilizing“split inteins”). A basic COMET toolkit comprises a suite of engineered proteins that regulate gene expression, including both activation and suppression of gene expression, and engineered DNA sequences that are regulated by these engineered proteins. Both the proteins and the cognate DNA sequences are modular in design, enabling one to tune the quantitative performance of the system and to multiplex these elements to build sophisticated, customized, cellular functions. The incorporation of split parts that can aid in the splicing or dimerization of the engineered proteins, thus improving the COMET system by adding a layer of post-translation control that was previously unutilized.

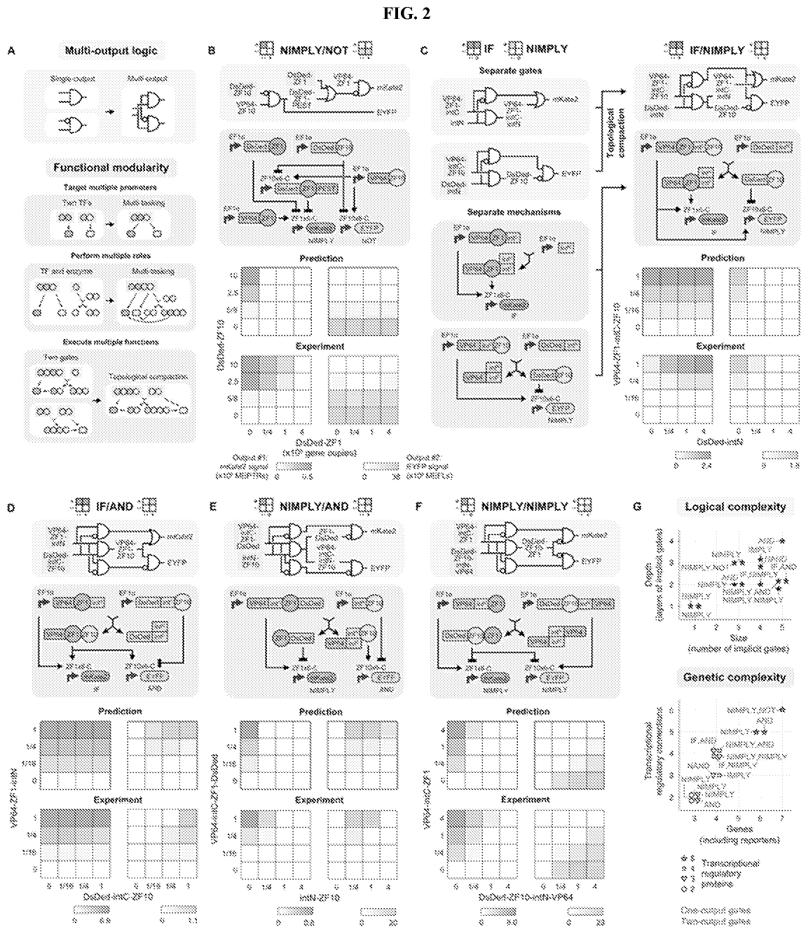

Thus, the present disclosure provides a platform for accurate genetic program design by engineering new parts (e.g., “split parts” utilizing “split inteins”) that combine transcriptional and post-translational control and which are validated by a computational modeling framework. The experimental observations disclosed herein (see Examples 1-6, below) closely matched simulations, even in scenarios employing new proteins (including those with many domains) and new topologies (including those with many interacting parts), demonstrating a high predictive capacity across a range of complexity (FIG. 2G). Since the mechanisms employed for binding, splicing, activation, and inhibition can be described by concise formalisms (see Example 1—Materials and Methods), no fundamental revamping (i.e., changing the underlying representation or granularity) of the disclosed descriptive model was needed to enable predictions. Furthermore, no trial-and-error (e.g., empirical tuning of designs or substitution of parts) was needed to arrive at the specified design goals, which streamlined the design-build-test-learn cycle. This is possible because once the base case parts were characterized, no additional parameterization was needed to simulate how the parts would function when combined in new designs. Lastly, even though a relatively small set of protein domains was utilized, the domains were able to be combined in many ways; i.e., a concise library was sufficient to produce the wide variety of behaviors observed.

The disclosed genetic circuits comprise an expression vector and one or more (e.g., 1, 2, 3, 4, 5, or more) engineered proteins which comprise at least two functional domains: (i) a DNA binding domain, (ii) a transcription modulation domain, which may activate or inhibit transcription of the expression vector. In general, the DNA binding domain of the one or more engineered proteins is capable of binding to a DNA binding site on the expression vector. In some embodiments, the expression vector may comprise a minimal promoter and a gene of interest, such as a reporter gene of some kind (e.g., a fluorescent protein or another detectable protein/peptide/signal). The presently disclosed genetic circuits are unique compared to other platforms because the engineered proteins of the present system comprise at least one or one or more (e.g., 1, 2, 3, 4, 5, or more) split inteins. Split inteins are short peptide elements comprising complementary domains that fold and trans-splice to covalently ligate flanking domains. Accordingly, the incorporation of split inteins into the one or more engineered proteins allows for post-translational modification of the engineered proteins in response to stimuli from the system in which the genetic circuit is employed.

In some embodiments, the DNA binding domain of an engineered protein may comprise, for example, all of or a functional fragment of a zinc finger domain, such as ZF1, ZF2, ZF3, ZF4, ZFS, ZF6, ZF7, ZF8, ZF9, ZF10, ZF11, ZF12, ZF13, ZF14, or ZF15. In some embodiments, the DNA binding domain of an engineered protein may comprise, for example, all of or a functional fragment of a zinc finger protein comprising more than three DNA-binding domains, other classes of programmable DNA binding domains (e.g., transcription activator-like effector (TALE)), DNA binding domains derived from microbial proteins (e.g., tetR, 1acI, etc.), and/or Cas9 or variants of Cas9 and other Cas proteins, including catalytically inactive variants (e.g., dCas9).

In some embodiments, a transcription activator domain of an engineered protein may comprise, for example, all of or a functional fragment of Herpes simplex virus protein 16 (VP16), a synthetic tetramer of VP16 (VP64), nuclear factor (NF) kappa-B (p65) or a subunit thereof, heat shock transcription factor 1 (HSF1), replication and transcription activator (RTA) of the gamma-herpesvirus family, p53, acidic domains (also known as “acid blobs” or “negative noodles,” rich in D and E amino acids, present in Ga14, Gcn4 and VP16), glutamine-rich domains (which may comprise multiple repetitions like “QQQXXXQQQ,” like those present in transcription factor Sp1), proline-rich domains (which may comprise repetitions like “PPPXXXPPP,” like those present in c-jun, AP2, and October 2), isoleucine-rich domains (which may comprise repetitions of “IIXXII,” like those present in NTF-1), and/or multipartite activators, such as VP64-p65-Rta (i.e., “VPR”; see Chavez et al., Nat Methods. 2015 Apr.; 12(4): 326-328).

In some embodiments, a transcription inhibition/inhibitor domain of an engineered protein may comprise, for example, all of or a functional fragment of ZF, DsDed-ZF, KRAB, Polycomb complexes, any domain that can fulfill a similar function for inhibition as a bulky DsRed variant, any domain which sterically occludes recruitment of the RNA polymerase complex or accessory factors, and/or chromatin modification modalities including histone de-acetylation, histone methylation, etc. as reviewed in Beisel & Paro, Nature Reviews Genetics, 12:123-135 (2011).

In some embodiments, a split intein (or one or more split inteins) may be incorporated into an engineered protein between the DNA binding domain and the transcription modulation domain (e.g., transcription activation domain or transcription inhibition domain). In some embodiments, a split intein (or one or more split inteins) may be incorporated onto the N-terminus of an engineered protein. In some embodiments, a split intein (or one or more split inteins) may be incorporated onto the C-terminus of an engineered protein. In some embodiments, a split intein (or one or more split inteins) may be incorporated onto both the N-terminus and the C-terminus of an engineered protein.

Many split inteins are known in the art, including but not limited to, gp41-1, Npu DnaE, Ssp DnaE, Mtu RecA, Sce VMA, Ssp DnaB-SO, Ssp DnaB-S1, and Ssp GyrB-S11. Any of these known split inteins may be incorporated into an engineered protein for the purposes of the disclosed genetic circuits. In some embodiments, the split intein is or comprises gp41-1 or a sequence derived therefrom. In some embodiments, an engineered protein of the present disclosure may comprise one, two, or all of SEQ ID NOs: 1, 2, and/or 3, or an amino acid sequence that possesses at least about 70%, at least about 75%, at least about 80%, at least about 85%, at least about 90%, at least about 95%, at least about 96%, at least about 97%, at least about 98%, or at least about 99% identity to any one of SEQ ID NOs: 1, 2, and/or 3.

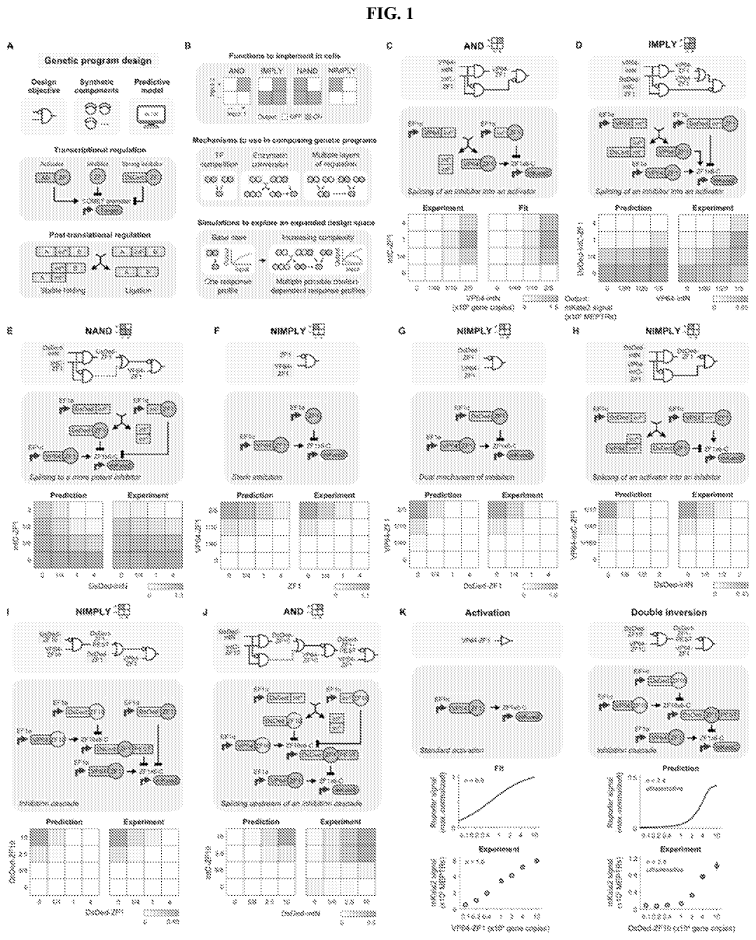

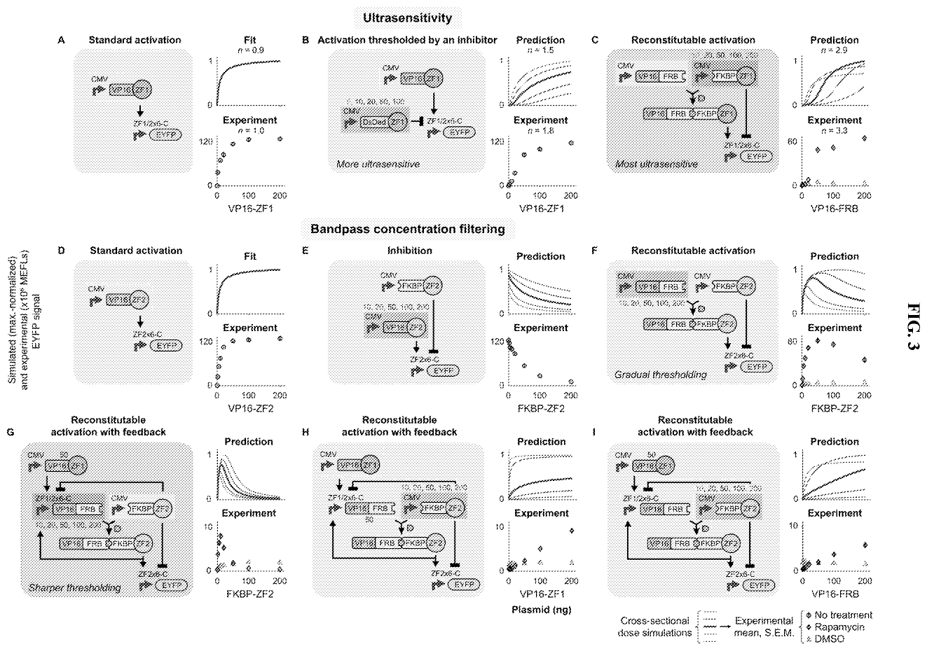

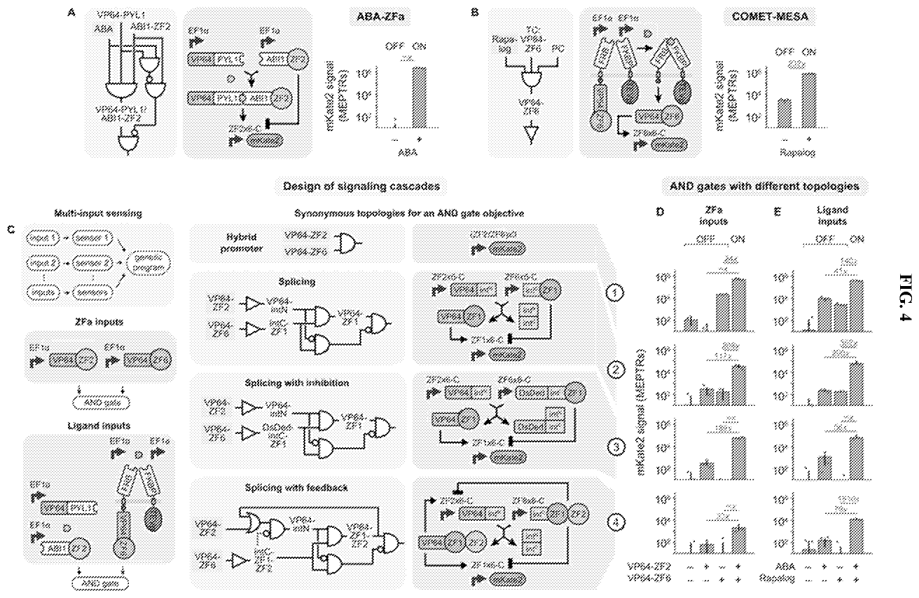

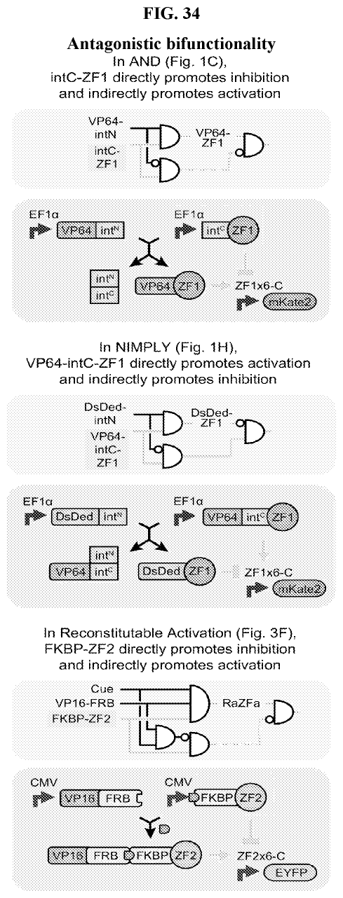

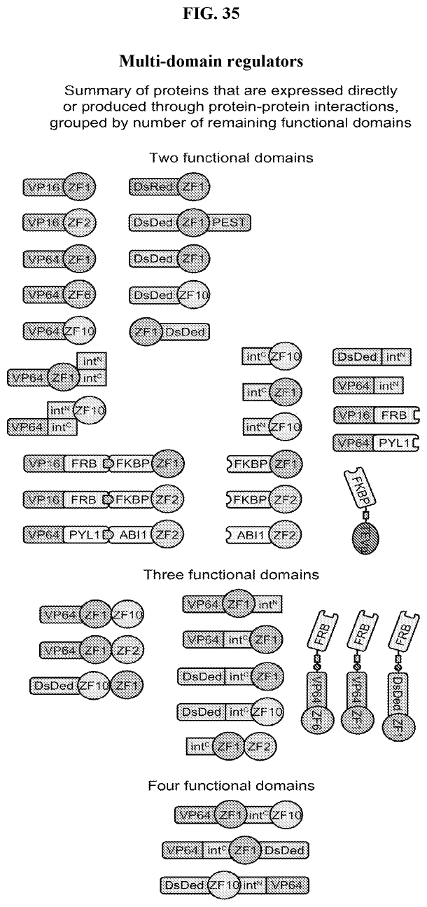

The disclosed strategy and examples can be used to prepare further circuit beyond those expressly disclosed. Key strategies that enabled sophisticated design included the use of antagonistic bifunctionality (48), in which a component can exert opposing effects on a target gene depending on the other components in the circuit (FIG. 34), and functional modularity, which enabled multiple activities to be combined in individual proteins (FIG. 35). Sophisticated design was also enabled through the use of split genetic parts, including those that splice or dimerize. Split parts are conducive to encoding both digital (FIGS. 1, 2) and analog (FIG. 3) functions. Split parts also shift some of the regulation from the transcriptional level to the post-translational level (i.e., protein-protein interactions), which could increase the speed of signal processing. Another benefit of split parts relates to circumventing cargo limitations of gene delivery vehicles, in that a large program that does not fit in one vector could be distributed across multiple vectors (49), in such a way that the parts interact to reconstitute the program only in cells receiving all of the vectors. Additionally, it was determined that seamless level-matching could be achieved with multi-layer circuits due to the potency of COMET TFs at cognate promoters, and in particular, the fusion of such a TF onto MESA resulted in the highest-performing version of this receptor to date (FIG. 4B,E).

The disclosed genetic circuit platform comprising split inteins may be integrated with previous described technology related to the use of Modular Expression Sensor Architecture (MESA). MESA technology is known in the art. (See e.g., Rachel M. Dudek, Ph.D. Dissertation entitled “Engineering Multiparametric Evaluation of Environmental Cues by Mammalian Cell-based Devices,” Northwestern University, August 2015; Daringer et al., “Modular Extracellular Sensor Architecture for Engineering Mammalian Cell-based Devices,” Nichole M. Daringer, Rachel M. Dudek, Kelly A. Schwarz, and Josh N. Leonard, ACS Synth. Biol. 2014, 3, 892-902, published Feb. 25, 2014; and international publication WO 2013/022739, published on Feb. 14, 2013; the contents of which are incorporated herein by reference in their entireties).

MESA systems typically include a pair of extracellular receptors where both receptors of the pair contain a ligand binding domain and transmembrane domain, and one receptor contains a protease cleavage site and a functional domain (e.g., transcription regulator such as a transcription regulator that promotes transcription or a transcription regulator that inhibits transcription) and the other receptor contains a protease domain. As used herein, a transcription regulator may include a transcription factor that promotes transcription (e.g., by recruiting additional cellular components for transcription) and/or a transcription inhibitor or transcription repressor). In some embodiments of the disclosed subject matter, a MESA receptor may comprise a transcription factor or transcription inhibitor as described herein for use in the technology platform as described herein.

The disclosed genetic circuit platform comprising split inteins may be integrated with previous technology related to the use of TANGO assays. (See Barnea et al., “The genetic design of signaling cascades to record receptor activation,” Proc Natl Acad Sci USA. 2008 Jan. 8; 105(1):64-69; the content of which is incorporated herein by reference in its entirety). In some embodiments of the disclosed subject matter, a TANGO assay and/or a receptor utilized in a TANGO assay may comprise a transcription factor or transcription inhibitor as described herein for use in the technology platform as described herein.

The disclosed genetic circuit platform comprising split inteins may be integrated with previous technology related to the use of synNotch assays. (See Morsut et al., “Engineering Customized Cell Sensing and Response Behaviors Using Synthetic Notch Receptors,” Cell. 2016 Feb. 11; 164(4): 780-791; the content of which is incorporated herein by reference in its entirety). In some embodiments of the disclosed subject matter, a synNotch pathway and/or a receptor utilized in a synNotch pathway may comprise or utilize a transcription factor or transcription inhibitor as described herein for use in the technology platform as described herein.

IV. Methods of Using the Disclosed Engineered Circuits

The present disclosure provides methods in which a host cell may be transiently or non-transiently transfected (i.e., stably transfected) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

The presently disclosed methods may include delivering one or more polynucleotides, such as or one or more vectors as described herein and/or one or proteins transcribed therefrom, to a host cell. Further contemplated are host cells produced by such methods, and organisms (such as animals, plants, or fungi) comprising or produced from such cells. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

V. Applications and Advantages of the Disclosed Platform

In one aspect, the disclosed compositions and models can be used in methods comprising mammalian cell-based therapies for treating diverse diseases including cancer, autoimmune disease, and metabolic diseases. In another aspect, the disclosed compositions and models can be used in methods of biomanufacturing using cells engineered to perform sophisticated functions and improve yield, quality, and efficiency of a biologic product. In another aspect, the disclosed compositions and models can be used in methods of gene therapy comprising delivery of compact genetic programs using parts/strategies described in this invention. In another aspect, the disclosed compositions and models can be used in methods of preparing stem cell-based products (e.g., for therapy or research) in which differentiation is controlled by a genetic program built using this technology platform.

Further applications of the disclosed technology platform may include, but are not limited to: (i) engineered cell-based therapies for cancer, autoimmune disease, regenerative medicine, and many other diseases; (ii) investigating fundamental biological questions (research), for example by expressing transgenes in mammalian cells at various levels or only under certain conditions; and (c) control of gene expression in biotechnology, for example production of recombinant proteins in mammalian cells.

The disclosed compositions and models enable construction of multi-tasking proteins, which carry out multiple transcriptional or post-translational activities, and enable the preparation/construction of genetically compact designs for executing complex operations, e.g., implementing two logic gates simultaneously. The disclosed compositions and models enable use of a modeling framework for designing genetic programs and predicting circuit performance based upon descriptions of the component parts, and the disclosed circuits are interoperable with existing technologies, e.g., they can be integrated with upstream COMET-compatible sensors.

This technology enables the construction of cell-based therapies and biomanufacturing platforms that perform functions not achievable with existing approaches. This capability enables the engineering of cell-based therapies that are safer and more effective (e.g., cancer treatments that better distinguish a tumor from healthy tissue to maximize treatment and minimize toxicity). This technology also enables the creation of novel types of therapeutic cells (e.g., stem cell-derived products that cannot be manufactured with existing protocols). This technology also enables the engineering of cells for biomanufacturing, such as for cells to scale up and adapt to culture conditions, or to control the timing/rate/yield of production of a biologic product.

Further advantages of the disclosed technology platform may include, but are not limited to: (i) the disclosed technology comprises a set of comparable transcription factors which recognize orthogonal binding sites and can therefore be multiplexed and used in combination to perform different tasks within a single cell; and (ii) many different parameters are readily tunable in the disclosed technology using either design-driven or experimentally identified variations in the engineered proteins and/or DNA sequences of the disclosed technology.

Altogether, the present disclosure greatly expands the mammalian genetic program design space. In the disclosed current system, one can propose and formulate models for candidate designs based on principles for how the functionally modular parts operate and then evaluate in silico outcomes. Indeed, it is possible to further automate this process by using software to sweep large combinatorial spaces and identify candidates that satisfy specified performance objectives. Such advances could further speed up the design process and broaden the scope of possible circuits and behaviors beyond those accessible solely by intuition. The new components and quantitative approaches developed here will enable bioengineers to build customized cellular functions for applications ranging from fundamental research to biotechnology and medicine.

The following examples are given to illustrate the present invention. It should be understood, however, that the invention is not to be limited to the specific conditions or details described in these examples.

### EXAMPLES

### Example 1—Materials and Methods

Experimental Method Details

Split Inteins

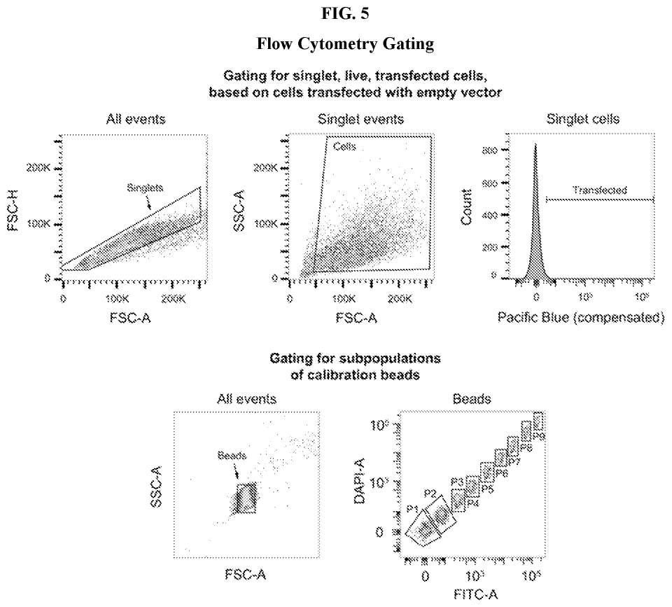

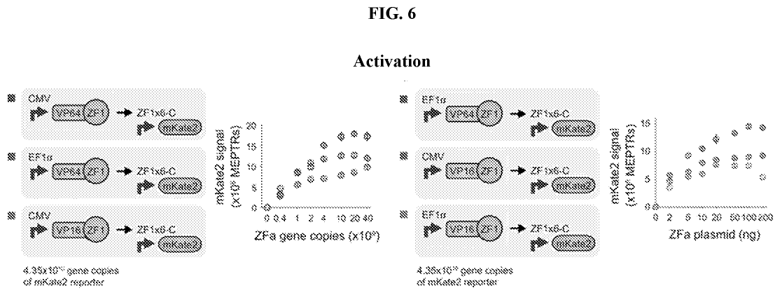

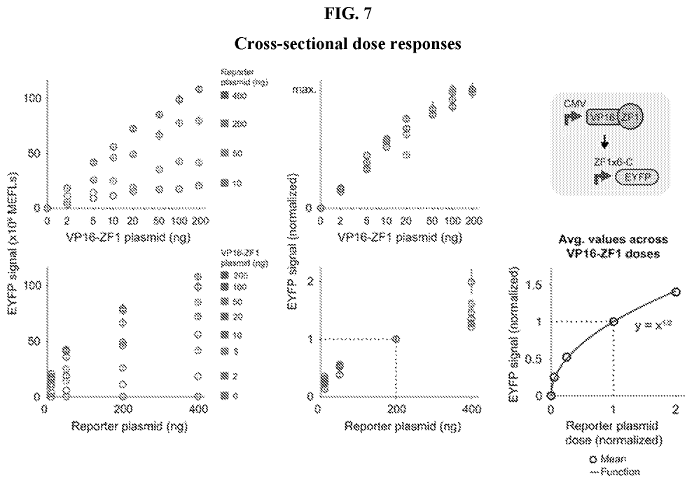

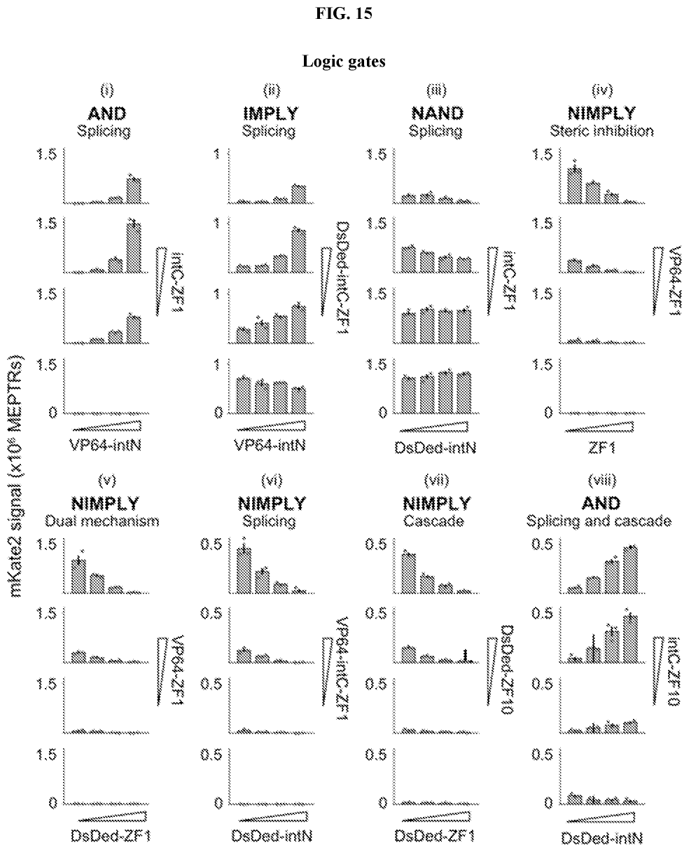

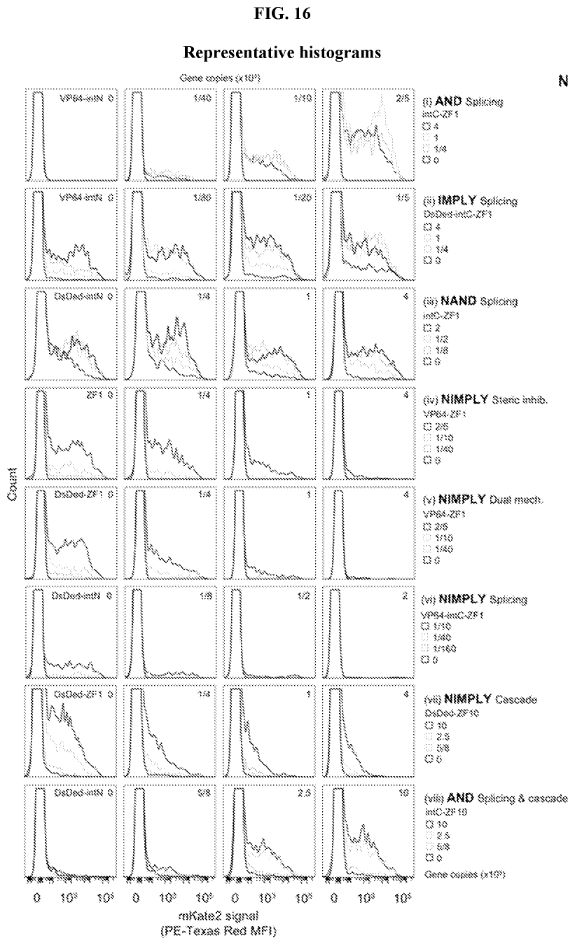

The COMET toolkit was expanded by incorporating gp41-1 (FIG. 1, FIGS. 5-17): a split intein that was identified putatively in a bioinformatic analysis (51), characterized in vitro and in E. coli (52), and later utilized in mammalian cells (53). The TRSGY (SEQ ID NO: 2) motif from the native sequence upstream of intN (at the end of the intN-adjoining extein) was included, as done by (52, 54-56) to retain high splicing activity; however, gp41-1 splicing has also been reported without this motif (53). It was important to use cysteine as the first amino acid of intN (“1” site) and serine as the first amino acid downstream of intC (“+1” site) (57).

The protein sequence for intN was:

(SEQ ID NO: 1)
CLDLKTQVQTPQGMKEISNIQVGDLVLSNTGYNEVLNVFPKSKKKSYKI
TLEDGKEIICSEEHLFPTQTGEMNISGGLKEGMCLYVKE, where
the first amino acid is the “1” site,
and
(SEQ ID NO: 2)
TRSGY preceded this site.

The protein sequence for intC was:

(SEQ ID NO: 3)
MMLKKILKIEELDERELIDIEVSGNHLFYANDILTHNS, where the
last amino acid is the “+1” site.

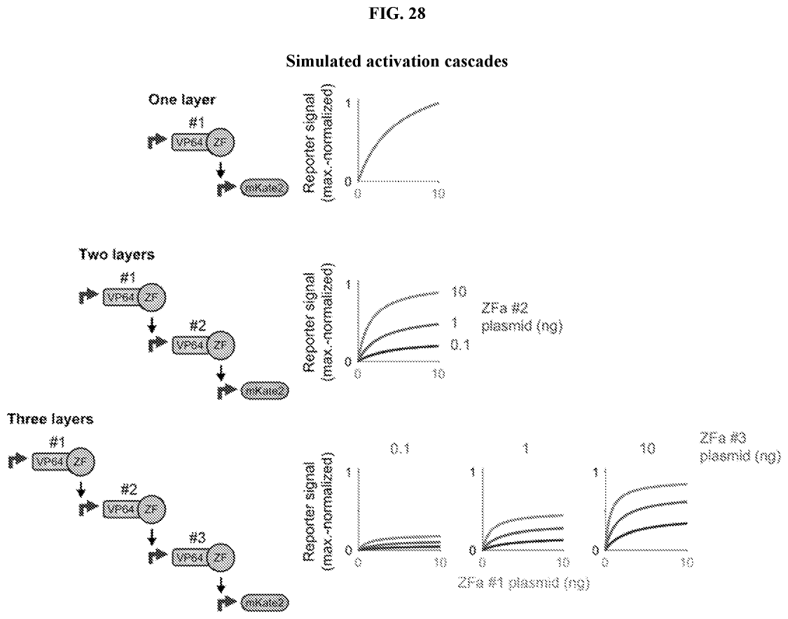

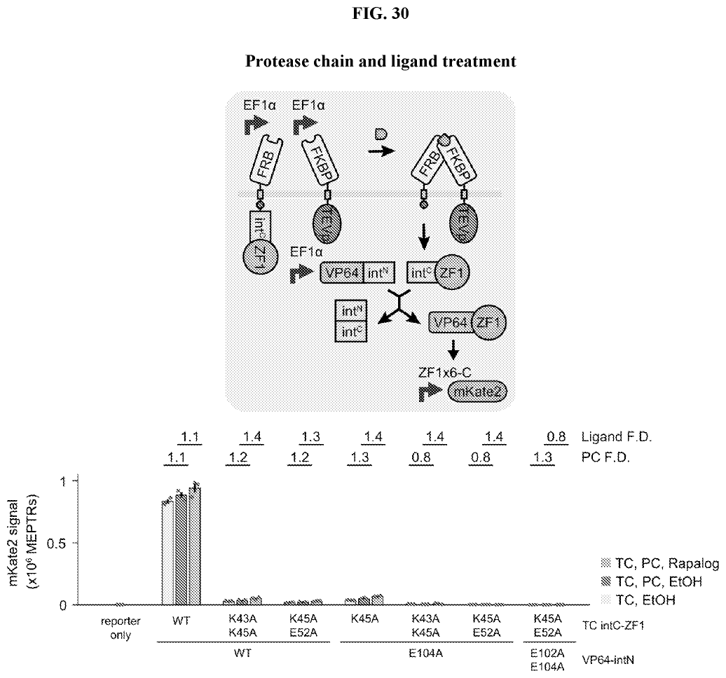

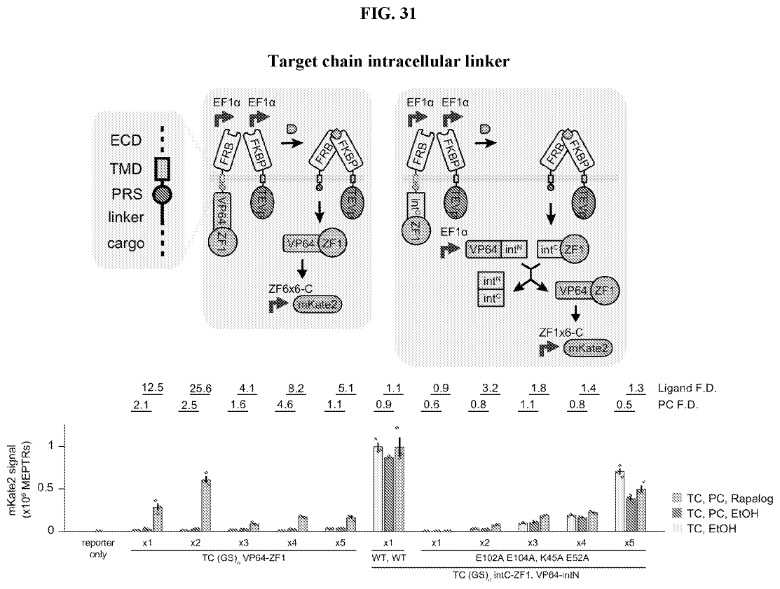

The mutagenesis investigation (FIGS. 28-35) was informed by a crystal structure of the gp41-1 C1A catalytically inactive mutant (58). Electrostatic interactions between the β3 strand at the end of intN and β6 strand at the start of intC were previously identified to form a charge zipper and proposed to be important in the capture and collapse mechanism that precedes splicing. In this mechanism, which was previously elucidated using the Npu DnaE split intein (59), capture involves electrostatic interactions between extended regions of the two fragments and collapse involves compaction and stabilization of their initially disordered regions.

Plasmid Cloning and Purification

Genetic components that were used in this study are listed in Table 1. Plasmids were designed in SnapGene (GSL Biotech LLC), and primers were ordered from Integrated DNA Technologies. Several domains were sourced from Donahue, et al. (60), but prior to the COMET study: VP16 and ZF domains are from Khalil, et al. (61), VP64 is from Chavez, et al. (Addgene #63798) (62), FRB and FKBP are from Daringer, et al. (Addgene #58876, #58877) (63), and DsRed-Express2 is a gift.

TABLE 1

| | | | |
|---|---|---|---|
| ID | Promoter | Gene | FIGS. |

Constitutive FPs

| | | | |
|---|---|---|---|
| pPD193 ⁱ | CMV | EBFP2 | 3-17, 25-35 |
| pPD133 ⁱ | CMV | EYFP | 3, 5-17, 25-27 |
| pJM409 | CMV | mKate2 | 5-17 |
| pJM413 | EF1α | mKate2 | 1, 2, 4-24, 28-35 |
| pJM451 | EF1α | EBFP2-P2A-BlastR | 5-17 |
| pJM500 | EF1α | EYFP | 2, 5-24 |
| pJM501 | EF1α | EBFP2 | 1, 2, 5-24, 28-35 |

Reporter FPs

| | | | |
|---|---|---|---|
| pPD270 ⁱ | ZF1/2x6-C | EYFP | 3, 25-27 |
| pPD290 ⁱ | ZF1x6-C | EYFP | 5-17 |
| pPD545 ⁱ | ZF2x6-C | EYFP | 3, 25-27 |
| pJM410 | ZF1x6-C | mKate2 | 1, 2, 4-24, 28-35 |
| pJM502 | ZF1x6-C | EYFP | 5-17 |
| pJM584 | ZF2x6-C | mKate2 | 4, 28-35 |
| pJM585 | ZF6x6-C | mKate2 | 4, 28-35 |
| pJM587 | ZF10x6-C | EYFP | 2, 18-24 |
| pJM597 | ZF(2/6)x3 | mKate2 | 4, 28-35 |

TFs (main group)

| | | | |
|---|---|---|---|
| pJB002 | CMV | 3xFLAG-NLS-DsDed-ZF1 | 3, 25-27 |
| pPD100 ⁱ | CMV | 3xFLAG-NLS-VP16-ZF1 | 3, 5-17, 25-27 |
| pPD189 ⁱ | CMV | 3xFLAG-NLS-VP16-ZF2 | 3, 25-27 |
| pPD303 ⁱ | CMV | 3xFLAG-NLS-VP64-ZF1 | 5-17 |
| pJM465 | EF1α | 3xFLAG-NLS-VP64-ZF1 | 5-17 |
| pJM466 | EF1α | 3xFLAG-NLS-VP16-ZF1 | 5-17 |
| pJM503 | EF1α | 3xFLAG-NLS-ZF1 | 1, 5-17 |
| pJM505 | EF1α | 3xFLAG-NLS-DsRed-ZF1 | 5-17 |
| pJM506 | EF1α | 3xFLAG-NLS-DsDed-ZF1 | 1, 2, 5-24 |
| pJM507 | EF1α | 3xFLAG-NLS-DsDed-ZF10 | 1, 2, 5-24 |
| pJM512 | EF1α | 3xFLAG-NLS-VP64-ZF1 | 1, 2, 5-24 |
| pJM520 | EF1α | 3xFLAG-NLS-VP64-ZF10 | 1, 2, 5-24 |
| pJM553 | ZF10x6-C | 3xFLAG-NLS-DsDed-ZF1-PEST | 1, 2, 5-24 |
| pJM574 | ZF2x6-C | 3xFLAG-NLS-DsDed-ZF1 | 4, 28-35 |
| pJM580 | ZF6x6-C | 3xFLAG-NLS-VP64-ZF1 | 4, 28-35 |
| pJM582 | EF1α | 3xFLAG-NLS-VP64-ZF2 | 4, 28-35 |
| pJM583 | EF1α | 3xFLAG-NLS-VP64-ZF6 | 4, 28-35 |

Small molecule-responsive TFs

| | | | |
|---|---|---|---|
| pJB001 | CMV | 3xFLAG-NLS-FKBP-ZF2 | 3, 25-27 |
| pJB003 | ZF1/2x6-C | 3xFLAG-NLS-VP16-FRB | 3, 25-27 |
| pPD341 ⁱ | CMV | 3xFLAG-NLS-FKBP-ZF1 | 3, 25-27 |
| pPD353 ⁱ | CMV | 3xFLAG-NLS-VP16-FRB | 3, 25-27 |
| pPD1122 | CMV | 3xFLAG-NES-PYL1-VP64 | 4, 28-35 |
| pKD011 | CMV | 3xFLAG-NLS-ZF2-ABI | 4, 28-35 |

TFs with WT split inteins

| | | | |
|---|---|---|---|
| pJB004 | CMV | 3xFLAG-NLS-VP16-intN | 25-27 |
| pJB005 | CMV | 3xFLAG-NLS-intC-ZF2 | 25-27 |
| pJM516 | EF1α | intC-ZF1-NLS-HA | 1, 5-17, 28-35 |
| pJM522 | EF1α | intC-ZF10-NLS-HA | 1, 5-17 |
| pJM524 | EF1α | 3xFLAG-NLS-VP64-intC-ZF1-NLS-HA | 1, 2, 18-24 |
| pJM525 | EF1α | 3xFLAG-NLS-DsDed-intC-ZF1-NLS-HA | 1, 5-17 |
| pJM529 | EF1α | 3xFLAG-NLS-DsDed-intC-ZF10-NLS-HA | 2, 18-24 |
| pJM554 | EF1α | 3xFLAG-NLS-VP64-intN | 1, 5-17, 28-35 |
| pJM555 | EF1α | 3xFLAG-NLS-DsDed-intN | 1, 2, 5-24 |
| pJM571 | ZF2x6-C | 3xFLAG-NLS-VP64-intN | 4, 28-35 |
| pJM572 | ZF6x6-C | intC-ZF1-NLS-HA | 4, 28-35 |
| pJM573 | ZF6x6-C | 3xFLAG-NLS-DsDed-intC-ZF1-NLS-HA | 4, 28-35 |
| pJM575 | ZF6x6-C | 3xFLAG-NLS-DsDed-ZF10 | 4, 28-35 |
| pJM576 | ZF2x6-C | 3xFLAG-NLS-VP64-intC-ZF1-NLS-HA | 4, 28-35 |
| pJM577 | ZF6x6-C | 3xFLAG-NLS-DsDed-intN | 4, 28-35 |
| pJM588 | EF1α | 3xFLAG-NLS-VP64-intC-ZF1-DsDed-NLS-HA | 2, 18-24 |
| pJM589 | EF1α | intN-ZF10-NLS-HA | 2, 18-24 |
| pJM590 | EF1α | 3xFLAG-NLS-VP64-ZF1-intC-ZF10-NLS-HA | 2, 18-24 |
| pJM591 | EF1α | 3xFLAG-NLS-VP64-ZF1-intN | 2, 18-24 |
| pJM592 | EF1α | 3xFLAG-NLS-DsDed-ZF10-intN-VP64 | 2, 18-24 |
| pJM595 | ZF6x6-C | intC-ZF1-ZF2-NLS-HA | 4, 28-35 |

TFs with mutant split inteins

| | | | |
|---|---|---|---|
| pJM556 | EF1α | 3xFLAG-NLS-VP64-intN_K43A | 28-35 |
| pJM557 | EF1α | 3xFLAG-NLS-VP64-intN_K45A | 28-35 |
| pJM558 | EF1α | 3xFLAG-NLS-VP64-intN_E52A | 28-35 |
| pJM559 ⁱⁱ | EF1α | 3xFLAG-NLS-VP64-intN_fiveA | 28-35 |
| pJM560 | EF1α | intC-ZF1-NLS-HA_E102A | 28-35 |
| pJM561 | EF1α | intC-ZF1-NLS-HA_E104A | 28-35 |
| pJM562 ⁱⁱⁱ | EF1α | intC-ZF1-NLS-HA_sixA | 28-35 |
| pJM563 | EF1α | 3xFLAG-NLS-VP64-intN_K43A_K45A | 28-35 |
| pJM564 | EF1α | 3xFLAG-NLS-VP64-intN_K45A_E52A | 28-35 |
| pJM565 | EF1α | intC-ZF1-NLS-HA_E98A_E104A | 28-35 |
| pJM566 | EF1α | intC-ZF1-NLS-HA_E102A_E104A | 28-35 |

Receptors (main group)

| | | | |
|---|---|---|---|
| pJM600 | EF1α | PC 3xFLAG-FKBP-FGFR4-TEVp | 4, 28-35 |
| pJM612 | EF1α | TC 3xFLAG-FRB-FGFR4-PRS(M)-(GS)x1- | 28-35 |
| | | NLS-VP64-ZF1 | |
| pJM621 | EF1α | TC 3xFLAG-FRB-FGFR4-PRS(M)-(GS)x1- | 28-35 |
| | | NLS-DsDed-ZF1 | |
| pJM671 | EF1α | TC 3xFLAG-FRB-FGFR4-PRS(M)-(GS)x1- | 4, 28-25 |
| | | NLS-VP64-ZF6 | |

Receptor with WT split intein and (GS)x1 linker

| | | | |
|---|---|---|---|
| pJM616 | EF1α | TC 3xFLAG-FRB-FGFR4-PRS(M)-(GS)x1-intC- | 28-35 |
| | | ZF1-NLS | |

Receptors with mutant split inteins and (GS)x1 linker

| | | | |
|---|---|---|---|
| pJM626 | EF1α | TC 3xFLAG-FRB-FGFR4-PRS(M)-(GS)x1-intC- | 28-35 |
| | | ZF1-NLS_E102A | |
| pJM627 | EF1α | TC 3xFLAG-FRB-FGFR4-PRS(M)-(GS)x1-intC- | 28-35 |
| | | ZF1-NLS_E104A | |
| pJM628 ⁱⁱⁱ | EF1α | TC 3xFLAG-FRB-FGFR4-PRS(M)-(GS)x1-intC- | 28-35 |
| | | ZF1-NLS_sixA | |
| pJM633 | EF1α | TC 3xFLAG-FRB-FGFR4-PRS(M)-(GS)x1-intC- | 28-35 |
| | | ZF1-NLS_E98A_E104A | |
| pJM634 | EF1α | TC 3xFLAG-FRB-FGFR4-PRS(M)-(GS)x1-intC- | 28-35 |
| | | ZF1-NLS_E102A_E104A | |

Receptors with extended (GS) linkers

| | | | |
|---|---|---|---|
| pJM614 | EF1α | TC 3xFLAG-FRB-FGFR4-PRS(M)-(GS)x2- | 28-35 |
| | | NLS-VP64-ZF1 | |
| pJM657 | EF1α | TC 3xFLAG-FRB-FGFR4-PRS(M)-(GS)x3- | 28-35 |
| | | NLS-VP64-ZF1 | |
| pJM658 | EF1α | TC 3xFLAG-FRB-FGFR4-PRS(M)-(GS)x4- | 28-35 |
| | | NLS-VP64-ZF1 | |
| pJM659 | EF1α | TC 3xFLAG-FRB-FGFR4-PRS(M)-(GS)x5- | 28-35 |
| | | NLS-VP64-ZF1 | |
| pJM661 | EF1α | TC 3xFLAG-FRB-FGFR4-PRS(M)-(GS)x2-intC- | 28-35 |
| | | ZF1-NLS_E102A_E104A | |
| pJM662 | EF1α | TC 3xFLAG-FRB-FGFR4-PRS(M)-(GS)x3-intC- | 28-35 |
| | | ZF1-NLS_E102A_E104A | |
| pJM663 | EF1α | TC 3xFLAG-FRB-FGFR4-PRS(M)-(GS)x4-intC- | 28-35 |
| | | ZF1-NLS_E102A_E104A | |
| pJM664 | EF1α | TC 3xFLAG-FRB-FGFR4-PRS(M)-(GS)x5-intC- | 28-35 |
| | | ZF1-NLS_E102A_E104A | |

Other

| | | | |
|---|---|---|---|
| pPD005 ⁱ | CMV | n/a | 1-35 |

ⁱ Generated and used in the COMET study (60)
ⁱⁱ Mutations in intN fiveA: K41A, K43A, K45A, K48A, E52A
ⁱⁱⁱ Mutations in intC sixA: K92A, K93A, E98A, E99A, E102A, E104A

Split inteins were from Hermann, et al. (Addgene #51267, #51268) (53). The ABA-binding domains PYL1 and ABI1 (64-68) from Gao, et al. (69) were utilized to make ABA-ZFa. The PEST tag was from the mouse ornithine decarboxylase gene (70). Two types of plasmid backbones were used: pcDNA (pPD005, Addgene #138749), which was modified from Thermo Fisher Scientific #V87020 as described by Donahue, et al. (60); and a series of transcription unit positioning vectors (TUPVs), which were derived from the modified pcDNA and previously published by Donahue, et al. (60), and based upon the mMoClo system from Duportet, et al. (71). Insulator sequences in TUPVs were from Bintu, et al. (Addgene #78099) (72).

Cloning was performed primarily using standard PCR, restriction, and ligation methods (reagents from New England Biolabs and Thermo Fisher Scientific), and in some cases through Golden Gate assembly, followed by transformation into chemically competent TOP10 E. coli (Thermo Fisher Scientific). Transformed E. coli were grown on LB/Ampicillin agar plates at 37° C., colonies were picked and grown in liquid LB/Ampicillin cultures, plasmid DNA was isolated (E.Z.N.A. plasmid mini kit, Omega Bio-tek), and DNA inserts were sequence-verified (ACGT, Inc.). Plasmids were prepared using polyethylene glycol-based extraction as described previously (60). DNA purity and concentration were measured using a Nanodrop 2000 (Thermo Fisher Scientific).

Mammalian Cell Culture

HEK293FT cells were cultured in complete DMEM medium containing 1% DMEM powder (Gibco #31600091), 0.35% w/v D-glucose (Sigma #50-99-7), 0.37% w/v sodium bicarbonate (Fisher #S233-500), 10% heat-inactivated FBS (Gibco #16140071), 4 mM L-glutamine (Gibco #25030081), and 100 U m⁻¹ penicillin and 100 μg ml⁻¹ streptomycin (Gibco #15140122) in tissue culture-treated 10 cm dishes (Corning #500001672) at 37° C. in 5% CO₂. To passage, medium was aspirated, and cells were washed in PBS, incubated in trypsin-EDTA (Gibco #25300054; 37° C., 5 min), detached by tapping the dish, and resuspended in fresh medium and plated. This cell line tested negative for Mycoplasma using the MycoAlert Mycoplasma detection kit (Lonza #LT07-318).

Transfection

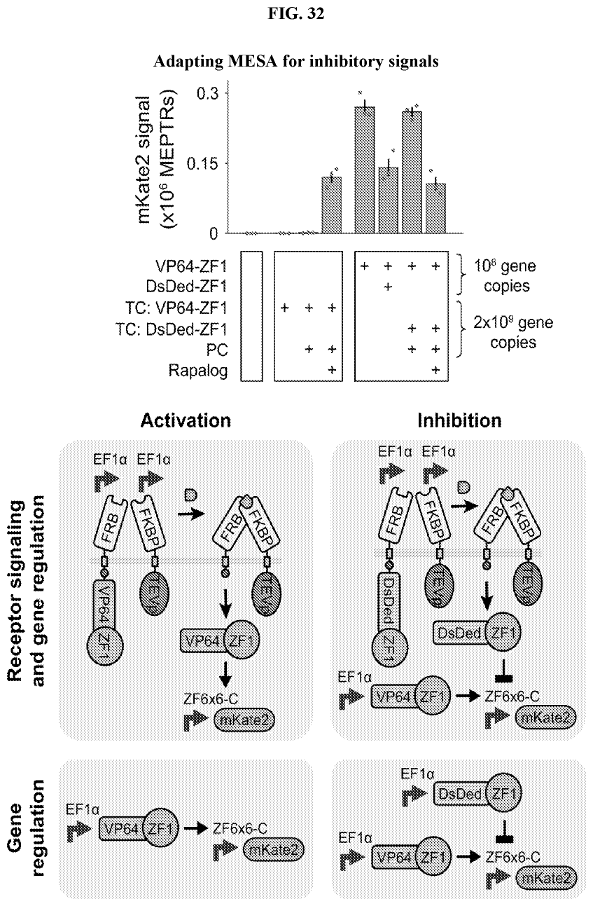

Cells were plated in 24-well plates (Corning #3524; 3×10⁵ cells ml⁻¹, 0.5 ml per well) and transfected after adhering to the plates, typically between 8-14 h after plating. Transfections were carried out using the calcium phosphate protocol (60): plasmids were mixed together in defined amounts, CaCl₂ (2 M, 15% v/v) was added, and this solution was pipetted dropwise into an equal volume of 2×HEPES-buffered saline (500 mM HEPES, 280 mM NaCl, 1.5 mM Na₂HPO₄); the solution was gently pipetted four times, and three minutes later it was vigorously pipetted 20 times and added dropwise onto plated cells. In this study, DNA doses are reported in plasmid mass (ng) per well of cells or gene copies per well of cells. In each transfection experiment, “empty vector” (pPD005) was included in the transfection mix to maintain a consistent total mass of DNA per well. At one day after plating, medium was aspirated and replaced with fresh medium. In some experiments, the fresh medium contained vehicle or ligand. In FIG. 3, the vehicle was 0.1% DMSO (v/v in cell culture) and the ligand was 100 nM rapamycin in 0.1% DMSO. In FIG. 4B, the vehicle was 0.1% EtOH and the ligand was either 100 nM rapalog (Takara #AP21697) or 100 μM abscisic acid (ABA; Goldbio #21293-29-8) in 0.1% EtOH. In FIG. 4D,E, the vehicle was 0.2% EtOH, and the ligand conditions included 100 nM rapalog, 100 μM ABA, or both ligands in 0.2% EtOH. For FIG. 32, treatment was applied both prior to transfection and at the time of medium change to promote more immediate inhibitory signaling.

Flow Cytometry

Samples were prepared for flow cytometry generally at 40-48 h post-transfection. For each well, medium was aspirated, five drops of PBS were added, PBS was aspirated, and two drops of trypsin-EDTA were added. Cells were incubated (37° C., 5 min), plates were tapped to detach cells, and four drops of cold (4° C.) DMEM were added. The contents of each well were pipetted up and down several times to detach cells and pipetted into FACS tubes containing FACS buffer (FB; 2 ml; PBS pH 7.4, 5 mM EDTA, 0.1% w/v BSA). Tubes were centrifuged (150×g, 5 min), liquid was decanted, and two drops of FB were added. Samples were kept on ice and wrapped in foil, and then run on a BD LSR Fortessa special order research product using the following configuration: Pacific Blue channel with 405 nm excitation laser and 450/50 nm filter for EBFP2; FITC channel with 488 nm excitation laser and 505LP 530/30 nm filter for EYFP; and PE-Texas Red channel with 552 nm excitation laser and 600LP 610/20 nm filter for mKate2. Approximately 10⁴ live single-cell events were collected per sample.

Flow Cytometry Data Analysis

Flow cytometry data were analyzed using FlowJo software (FlowJo, LLC) to gate on single-cell (FSC-A vs. FSC-H) and live (FSC-A vs. SSC-A) bases, compensated using compensation control samples, and gated as transfection-positive (FIG. 5). The mean reporter signal in MFI was obtained for each sample. UltraRainbow Calibration Particles (Spherotech #URCP-100-2H) were run in each flow cytometry experiment. Beads were gated on an FSC-A vs. FSC-H basis, the nine bead subpopulations of varying intensities were identified, and the mean MFI for each subpopulation in the FITC channel and PE-Texas Red channel was obtained. These values in combination with manufacturer-supplied MEFL and MEPTR values for each subpopulation were used to fit a regression line with y-intercept equal to zero. The mean and S.E.M. for the three biological replicates were calculated. Autofluorescence background signal was subtracted using samples transfected with the transfection control marker, and error was propagated. MFI values were converted to MEFL or MEPTR using the slope of the regression line, and error was propagated. Histograms in supplementary figure panels represent reporter signal in MFI.

Nomenclature

Genes were named by their protein domains in order from N-terminus to C-terminus. Domains were generally connected by flexible linkers comprising glycine and serine. Several abbreviations were used: ZFa was an AD-ZF for any choice of AD and ZF; similarly, RaZFa was an AD-FRB and FKBP-ZF, and ABA-ZFa was an AD-PYL1 and ABI1-ZF. DsRed refers to wild type DsRed-Express2, and DsDed was an DsRed-Express2 R95K mutant. This is a streamlined nomenclature that differs from that used in the original COMET report (60), in that inhibitors do not use ZFi notation: ZFi is now termed ZF, and DsRed-ZFi is now termed DsRed-ZF.

The constitutive promoters used were CMV and EF1α. The inducible promoters used were COMET promoters, which were named as “[ZF domain]x[number of binding sites]-[binding site arrangement]”. For example, ZF1x6-C has six compact sites for ZF1. There were two non-standard cases: ZF1/2x6-C has six compact overlapping sites for ZF1 and ZF2 (up to six sites occupied, and up to six per ZF); (ZF2/ZF6)x3 has six compact sites alternating between ZF2 and ZF6 (up to six sites occupied, and up to three per ZF).

Statistical Analysis

Each sensor in FIG. 4A,B was assessed using a one-tailed Welch's unpaired t-test, with the null hypothesis that reporter signal was equal with and without ligand treatment. Genetic programs in FIG. 4D,E were assessed using a three-factor ANOVA and Tukey's honest significant difference (HSD) test, with the null hypothesis that reporter signal was equal across the two input types, four topologies, and four input combinations. Effects were considered significant if p<0.05, and additionally for the HSD test if the comparisons had an adjusted p<0.05.

Computational Method Details

Overview

This section describes the extension of another explanatory model for COMET TFs (60) to a predictive model incorporating split intein-mediated splicing and other attributes. Rules for formulating systems of ODEs are provided to support the formulation of models for new genetic circuits based on the genetic parts from this study.

A Statistical Model for Gene Expression Heterogeneity

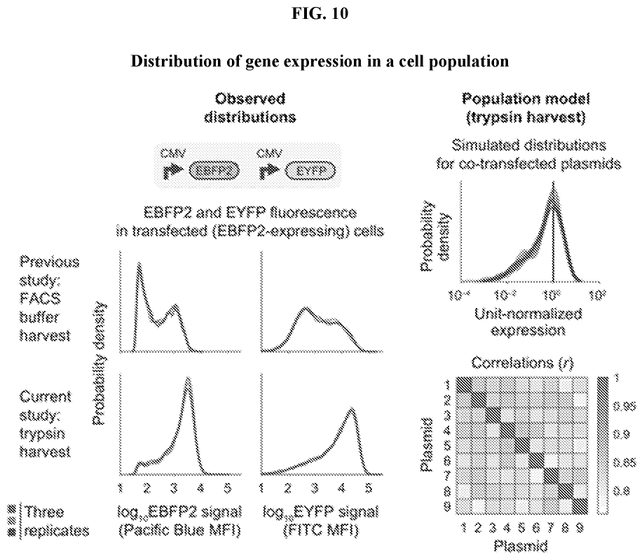

This modeling approach accounts for variation in gene expression—including differences in the expression of a gene between cells and differences in the expression of genes within a cell—in a cell population. A population matrix was generated using the constrained sampling method (60, 73), which was used here to describe the distribution of gene expression observed when cells were harvested via trypsin digest (as noted in FIG. 10, this distribution differs somewhat from the distribution observed when cells were harvested by another common method, suspension in FACS buffer).

The ith row (cell) and pth column (plasmid) of the population matrix Z was a scalar for the relative expression of a gene. The z value was used as a multiplier in the production term for each RNA species.

Production rate=Z_{i,p}·k_{txEF1a}·dose (1)

A dynamical model was run separately for each cell in the simulated population, and the mean end-point simulated reporter protein level for the population was calculated. Layering the statistical model on the dynamical models at the level of RNA production enables the simulations to account for cell-to-cell variation (e.g., this method incorporates potential outlier effects that could skew a population mean) and therefore should generally enable better predictions (73).

Some figures employ a standard single-cell (homogeneous) model, as indicated in the figure description. These cases forgo the incorporation of heterogeneity and instead simulate the mean-transfected cell, which represents a scenario for average gene expression from each plasmid.

Dynamical Models

Genetic programs were represented by systems of ODEs. State variables included RNA and protein species in arbitrary concentration units. Processes included transcription (constitutive, inducible, inhibitable), RNA degradation, protein translation, split intein-mediated splicing, small molecule-based reconstitution, and protein degradation. Parameter values are in Table 2.

TABLE 2

| Symbol | Description | Value ^{i,ii} | | Source |
|---|---|---|---|---|
| b₁ | Basal transcription at ZF1x6-C promoter | 0.08 | U | COMET |
| m₁ | Max. induction for CMV-driven VP16-ZF1 at ZF1x6-C | 33 | | COMET |
| | promoter | | | |
| w₁ | Steepness for CMV-driven VP16-ZF1 at ZF1x6-C | 0.036 | | Fitted here |
| | promoter | | | |
| m_{1E64} | Max. induction by EF1α-driven VP64-ZF1 at ZF1x6-C | 52 | | Fitted here |
| | promoter | | | |
| w_{1E64} | Steepness for EF1α-driven VP64-ZF1 at ZF1x6-C | 0.192 | | Fitted here |
| | promoter | | | |
| b₂ | Basal transcription at ZF2x6-C promoter | 0.25 | U | COMET |
| m₂ | Max. induction by CMV-driven VP16-ZF2 at ZF2x6-C | 54 | | COMET |
| | promoter | | | |
| w₂ | Steepness for CMV-driven VP16-ZF2 at ZF2x6-C | 0.082 | | Fitted here |
| | promoter | | | |
| b_{H} | Basal transcription at ZF1/2x6-C promoter | b₁ | | Assumed |
| m_{1H} | Max. induction by CMV-driven VP16-ZF1 at ZF1/2x6- | m₁ | | Assumed |
| | C promoter | | | |
| m_{2H} | Max. induction by CMV-driven VP16-ZF2 at ZF1/2x6- | m₂ | | Assumed |
| | C promoter | | | |
| w_{1H} | Steepness for CMV-driven VP16-ZF1 at ZF1/2x6-C | 0.072 | | Fitted here |
| | promoter | | | |
| w_{2H} | Steepness for CMV-driven VP16-ZF2 at ZF1/2x6-C | 0.170 | | Fitted here |
| | promoter | | | |
| b₁₀ | Basal transcription at ZF10x6-C promoter | 0.01 | U | COMET |
| m_{10E64} | Max. induction by EF1α-driven VP64-ZF10 at ZF10x6- | m_{1E64} | | Assumed |
| | C promoter | | | |
| w_{10E64} | Steepness for EF1α-driven VP64-ZF10 at ZF10x6-C | w_{1E64} | | Assumed |
| | promoter | | | |
| l | Start of loss of cooperativity (0.5 in COMET) | 0 | | Adjusted here |
| u | End of loss of cooperativity (2 in COMET) | 1.5 | | Adjusted here |
| w_{r1E64} | Steepness for DsDed-ZF1 inhibition of EF1α-driven | 4 * w_{r1E64} | | Definition |
| | VP64-ZF1 at ZF1x6-C | | | |
| w_{r10E64} | Steepness for DsDed-ZF10 inhibition of EF1α-driven | 4 * w_{r10E64} | | Definition |
| | VP64-ZF10 at ZF10x6-C | | | |
| w_{r1H} | Steepness for DsDed-ZF1 inhibition of CMV-driven | 4 * w_{r1H} | | Definition |
| | VP64-ZF1 at ZF1/2x6-C | | | |
| rec | Reconstitution of split TFs (fitted based on split inteins; | 0.34 | U⁻¹ h⁻¹ | Fitted here |
| | also applied to RaZFa) | | | |
| k_{txCMV} | Transcription at CMV promoter | 1 | U h⁻¹ | Default |
| k_{txEF1a} | Transcription at EF1α promoter | 1 | U h⁻¹ | Default |
| k_{txZF} | Transcription at COMET promoters | 1 | U h⁻¹ | Assumed |
| kₜₗ | Translation | 1 | h⁻¹ | Default |
| k_{degR} | Degradation of RNA | 2.7 | h⁻¹ | COMET |
| k_{degZFP} | Degradation of TF protein (default) | 0.35 | h⁻¹ | COMET |
| k_{degZFP}_{_{—}}_{PEST} | Degradation of PEST-tagged TF protein | 0.7 | h⁻¹ | Assumed |
| k_{degintC} | Degradation of intC-containing TF protein | 1.3 | h⁻¹ | Fitted here |
| k_{degRep} | Degradation of reporter protein | 0.029 | h⁻¹ | COMET |

Constitutive transcription from the EF1α promoter or CMV promoter was proportional to plasmid dose (ng).

RNA production rate=k_{txEF1a}·dose (2)

RNA production rate=k_{txCMV}·dose (3)

Functions for regulated transcription were broadly represented by f The dose term d for a regulated gene was empirically defined (i.e., based on a heuristic) and calculated by dividing the plasmid dose (ng) by 200 ng; then, the square root of this fraction was used, e.g., for 200 ng, d^{1/2}=1, and for 50 ng, d^{1/2}=0.5. The 200 ng dose was defined as a reference point because this was the dose of reporter plasmid used in the original characterization (60).

RNA production rate=k_{txZF}·d^{1/2}·f (4)

ZFa-inducible transcription uses the COMET model formulation, in which b is TF-independent (background) transcription, m is the maximal activation, and w is a steepness parameter. Also modeled was the activation mediated by AD-ZF-containing proteins that also contain intC, intN, or additional ZF domains equivalently to that by a base case ZFa. The variable refers to the simulated amount of TF protein, not to plasmid dose.

$$f = \frac{b + m \cdot w \cdot {ZFa}_{Protein}}{1 + w \cdot {ZFa}_{Protein}} \qquad ( 5 )$$

ZFa-inducible transcription can be inhibited by a ZF, which sterically blocks the activator from binding to sites in a promoter. Also modeled was the inhibition mediated by ZF proteins that also contain intC, intN, FKBP, or additional ZF domains equivalently to that by a base case ZF. The subscripts A and I denote parameters for an activator and inhibitor, respectively.

$$f = \frac{b + {m}_{A} \cdot {w}_{A} \cdot {ZFa}_{Protein}}{1 + {w}_{A} \cdot {ZFa}_{Protein} + {w}_{I} \cdot {ZF}_{Protein}} \qquad ( 6 )$$

ZFa-inducible transcription can also be inhibited by a DsDed-ZF, which acts through a dual mechanism of steric inhibition and reduction of effective promoter cooperativity. The effect of the latter mechanism is that at increasing strength or dose of inhibitor compared to activator, the cooperativity represented by m ramps down to an effective value of 1. Also modeled was the inhibition mediated by DsDed-ZF-containing proteins that also contain intC, intN, or additional ZF domains equivalently to that by a base case DsDed-ZF.

$$f = \frac{\begin{matrix}b + max ( min ( \frac{( \frac{4 \cdot {w}_{I} \cdot DsDed - {ZF}_{Protein}}{{w}_{A} \cdot {ZFa}_{Protein}} ) - 4 \cdot l ) ( 1 - m )}{4 \cdot u - 4 \cdot l} + \\ m , m ) , 1 ) \cdot {w}_{A} \cdot {ZFa}_{Protein}\end{matrix}}{1 + {w}_{A} \cdot {ZFa}_{Protein} + 4 \cdot {w}_{I} \cdot DsDed - {ZF}_{Protein}} \qquad ( 7 )$$

RNA degradation is represented as a first-order process.

RNA degradation rate=−k_{degRNA}·Species_{RNA} (8)

Protein translation is also first order.

Translation rate=kₜ₁·Species_{RNA} (9)

Splicing is a second-order reaction between an intN-containing protein and an intC-containing protein with a fitted rate constant.

Splicing rate=k_{rec}·Species1_{Protein}·Species2_{Protein} (10)

For example, the following the terms represent the splicing of A-intN-B and X-intC-Y to A-Y and X-intC/intN-B, where A, B, X, and Y can be DNA-binding, activating, or inhibitory domains or no domain.

A-intN-B_{Protein} splicing rate=−k_{rec}·A-intN-B_{Protein}·X-intC-Y_{Protein} (11)

X-intC-Y_{Protein} splicing rate=−k_{rec}·A-intN-B_{Protein}·X-intC-Y_{Protein} (12)

A-Y_{Protein} splicing rate=k_{rec}·A-intN-B_{Protein}·X-intC-Y_{Protein} (13)

X-intC/intN-B_{Protein} splicing rate=k_{rec}·A-intN-B_{Protein}·X-intC-Y_{Protein} (14)

Small molecule-based reconstitution to form RaZFa uses the Heaviside function H with ligand treatment at time τ (hours) post-transfection. For simplicity, the k_{rec} parameter was also used to describe reconstitution.

RaZFa reconstitution rate=k_{rec}·AD-FRB_{Protein}·FKBP-ZF_{Protein}·H(t−τ) (15)

Prior to reconstitution, FKBP-ZF can act as a ZF-like inhibitor against RaZFa or ZFa at a target promoter.

$$RNA production rate = {k}_{txZF} \cdot \frac{b + m \cdot w \cdot {RaZFa}_{Protein}}{1 + w \cdot {RaZFa}_{Protein} + w \cdot FKBP - {ZF}_{Protein}} \qquad ( 16 )$$

Protein degradation is first order. Rate constants vary for non-intC-containing TFs, intC-containing TFs, and reporter protein, respectively.

Protein degradation rate=−k_{degZFP}·TF_{Protein} (17)

Protein degradation rate=−k_{degintC}·TF_{Protein} (18)

Protein degradation rate=−k_{degRep}·Reporter_{Protein} (19)

As an example, the following system of equations represents the reconstitution of a ZFa and induction of a reporter. This system produces an AND gate for: if AD-intN and intC-ZF are present, then induce reporter.

$$\begin{matrix}\frac{dAD - {intN}_{RNA}}{dt} = {z}_{i , 1} \cdot {k}_{txEF 1 a} \cdot {dose}_{AD - intN} \\ - {k}_{degRNA} \cdot AD - {intN}_{RNA}\end{matrix} \qquad ( 20 )$$

$$\begin{matrix}\frac{dAD - {intN}_{Protein}}{dt} = {k}_{tl} \cdot AD - {intN}_{RNA} \\ - {k}_{rec} \cdot AD - {intN}_{Protein} \cdot intC - {ZF}_{Protein}\end{matrix} \qquad ( 21 )$$

$$\begin{matrix}\frac{dintC - {ZF}_{RNA}}{dt} = - {k}_{degZFP} \cdot AD - {intN}_{Protein} \\ {z}_{i , 2} \cdot {k}_{txEF 1 a} \cdot {dose}_{intC - ZF} \\ - {k}_{degRNA} \cdot intC - {ZF}_{RNA}\end{matrix} \qquad ( 22 )$$

$$\begin{matrix}\frac{dintC - {ZF}_{Protein}}{dt} = {k}_{tl} \cdot intC - {ZF}_{RNA} \\ - {k}_{rec} \cdot AD - {intN}_{Protein} \cdot intC - {ZF}_{Protein} \\ - {k}_{degintC} \cdot intC - {ZF}_{Protein}\end{matrix} \qquad ( 23 )$$

$$\begin{matrix}\frac{dAD - {ZF}_{Protein}}{dt} = {k}_{rec} \cdot AD - {intN}_{Protein} \cdot intC - {ZF}_{Protein} \\ - {k}_{degZFP} \cdot AD - {ZF}_{Protein}\end{matrix} \qquad ( 24 )$$

$$\begin{matrix}\frac{dintC / {intN}_{Protein}}{dt} = {k}_{rec} \cdot AD - {intN}_{Protein} \cdot intC - {ZF}_{Protein} \\ - {k}_{degintC} \cdot intC / {intN}_{Protein}\end{matrix} \qquad ( 25 )$$

$$\begin{matrix}\frac{{dReporter}_{RNA}}{dt} = {z}_{i , 3} \cdot {k}_{txZF} \cdot {d}_{Reporter}^{1 / 2} \cdot \\ \frac{b + m \cdot w \cdot AD - {ZF}_{Protein}}{1 + w \cdot AD - {ZF}_{Protein} + w \cdot intC - {ZF}_{Protein}} \\ - {k}_{degRNA} \cdot {Reporter}_{RNA}\end{matrix} \qquad ( 26 )$$

$$\begin{matrix}\frac{{dReporter}_{Protein}}{dt} = {k}_{tl} \cdot {Reporter}_{RNA} \\ - {k}_{degRep} \cdot {Reporter}_{Protein}\end{matrix} \qquad ( 27 )$$

Since the genetic parts employed for activation, inhibition, splicing, and dimerization exhibit functional modularity, one can utilize the formalisms described above to generate systems of equations to represent a variety of circuits. ODEs for the circuits in this study were provided in MATLAB files.

Parameterization

Some parameter values are from the COMET study (60) and others are newly estimated or fitted here (Table 2).

Ultrasensitivity

Ultrasensitivity is a type of nonlinear signal processing in which a small change in an input produces a large change in an output. It was demonstrated how this property can be achieved with engineered motifs such as a double inhibition cascade (FIG. 1K), activation thresholded by an inhibitor (FIG. 3B), and reconstitutable activation (FIG. 3C). The ultrasensitivity of experimental and simulated dose responses was quantified using the Hill coefficient n from a modified Hill equation, in which x is input plasmid dose (ng), y is reporter signal (MEPTRs or MEFLs), y₀ is reporter signal for zero input, and a and b are other fitted parameters. Standard ZFa dose responses are characterized by n˜1. Ultrasensitive responses are those for which n>1.

$$y = {y}_{0} + \frac{a \cdot {x}^{n}}{{( \frac{1}{b} )}^{n} + {x}^{n}} \qquad ( 28 )$$

Diagrams

Genetic programs for digital functions are depicted using genetic diagrams and electronic diagrams. The former represents each promoter, protein, and regulatory interaction, and the latter represents the logic underlying these interactions.

### Example 2—Biological Parts for Integrating Transcriptional and Post-Translational Control of Gene Expression

The strategy that was pursued for genetic program design was uniquely enabled by the COmposable Mammalian Elements of Transcription (COMET): a toolkit of TFs and promoters with tunable properties enabling precise and orthogonal control of gene expression (13). These TFs comprised a ZF DNA-binding domain and a functional domain, e.g., VP16 and VP64 are activation domains (AD) that in combination with a ZF form an activator (ZFa). A protein including a ZF domain but lacking an AD can function as a competitive inhibitor of the cognate ZFa. Promoters in this library contained ZF binding sites arranged in different configurations (e.g., ZF1x6-C has six compactly arranged ZF1 sites). Each combination of a promoter and a ZFa (and potentially an inhibitor) conferred a characteristic level of transcriptional activity (FIGS. 5-7), and as part of this work, mathematical models were developed to characterize these relationships (13). Here, the investigation focused on whether these biological parts and descriptive computational tools could be adapted and applied to achieve predictive genetic program design.

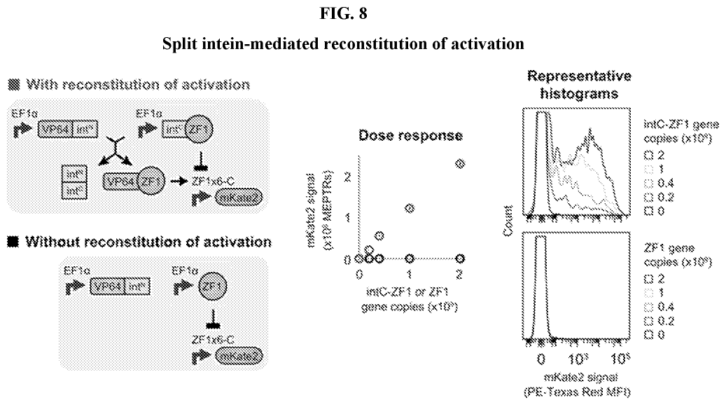

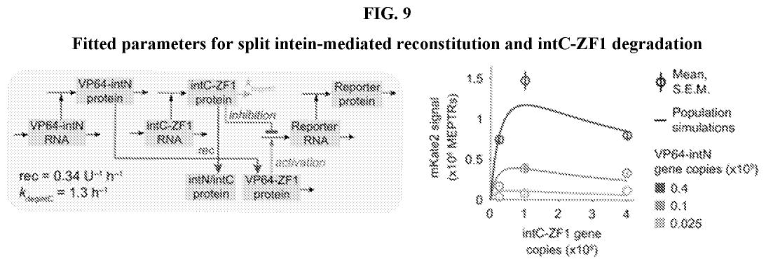

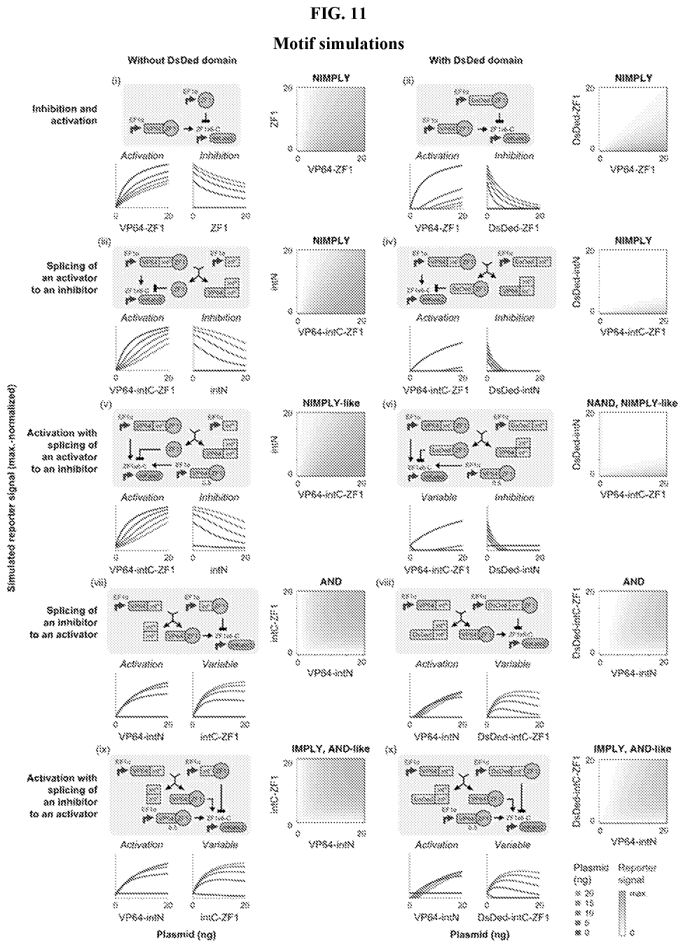

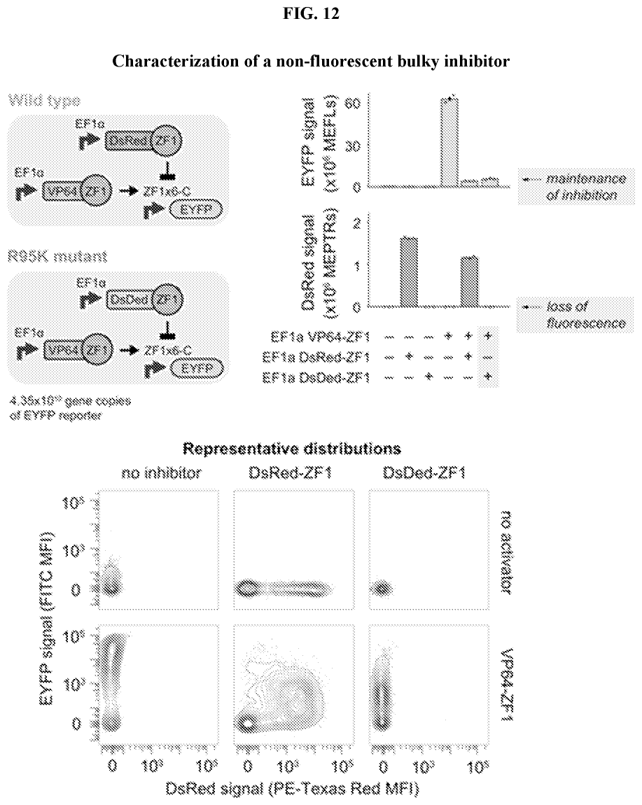

Although COMET includes many parts for implementing transcriptional regulation, complex genetic program design was facilitated by introducing a mechanism for regulation at the post-translational level (FIG. 1A,B). To investigate this strategy, experiments were developed to evaluate new parts based on split inteins: complementary domains that fold and trans-splice to covalently ligate flanking domains (exteins) (29). The split intein gp41-1 was selected for its rapid splicing kinetics (30). To test an application of this mechanism, an AD was appended to the gp41-1 N-terminal fragment (intN) and a ZF to the C-terminal fragment (intC). These parts were used to construct an AND gate in which a reporter gene was induced only when both fragments were present (FIG. 1C, FIG. 8), thus demonstrating that COMET-mediated gene expression can be adapted with splicing. Next, this mechanism was incorporated into a modeling framework by modifying ordinary differential equations from a COMET study (13), which concisely represented transcriptional regulation (see Example 1—Materials and Methods), and fitted newly introduced parameters to the data (FIGS. 9-10). The model was extended to describe parts in which split inteins were fused onto two types of inhibitors (FIG. 11): ZF, which competes with ZFa for binding site occupancy in the promoter; and ZF fused to DsRed-Express2 (abbreviated as DsRed-ZF), which also reduced the cooperativity of ZFa-mediated RNA polymerase II (RNAPII) recruitment at multi-site promoters (13). Additionally, an R95K mutation was introduced to ablate the DsRed chromophore (31), yielding a non-fluorescent inhibitor that was termed DsDed-ZF (FIG. 12). The extended model accurately recapitulated the component dose-dependent performance of the AND gate (FIG. 1C), providing verification that this extension can describe split intein-based circuits.

### Example 3—Model-Guided Design of Genetic Programs

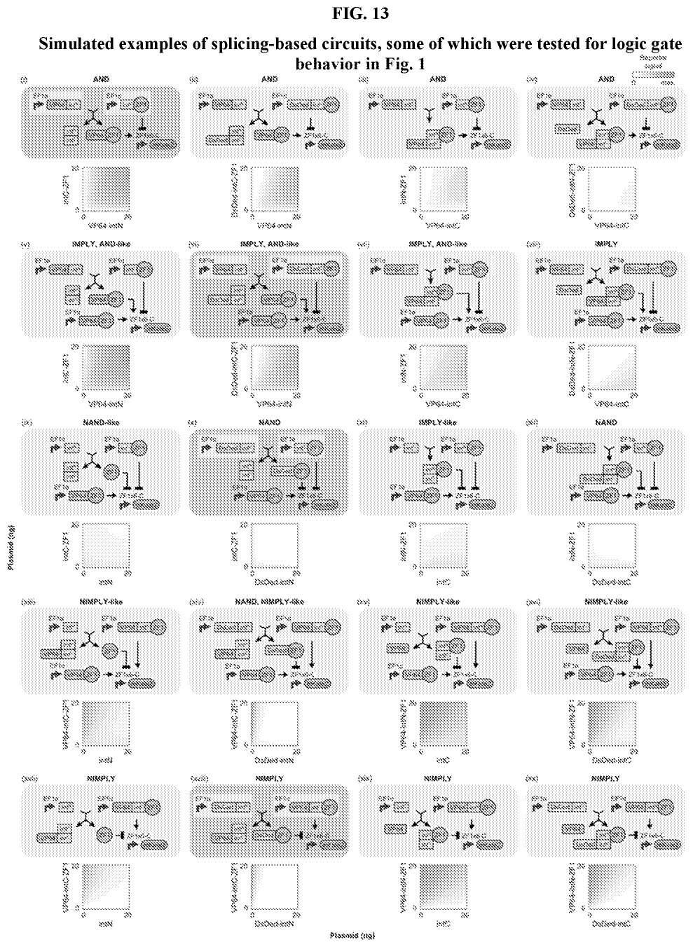

As a first test of the predictive capacity of the revised model, a panel of circuits that could carry out various logic operations was simulated (FIG. 13). The objective was to identify promising designs for specific functions, so no additional model complexity that might be required to predict all aspects of circuit behavior (e.g., potential cell burden effects)was included. Throughout, simulations employed a statistical model for gene expression variation, which was previously shown to be important in accounting for the effect of cellular variation on how an engineered function is carried out across a cell population (13, 32) (Example 1—Materials and Methods, FIG. 10). From the panel, several designs were selected to test. First, to make an IMPLY gate, the AND gate was modified by appending DsDed to intC-ZF1 and co-expressing a VP64-ZF1 activator. Experimental outcomes (i.e., reporter readout across component doses) were consistent with the prediction that readout would be low only with DsDed-intC-ZF1 present in sufficient excess over its VP64-intN splicing partner to function as an inhibitor (FIG. 1D). To make a NAND gate, a DsDed-ZF1 inhibitor was split into DsDed-intN and intC-ZF1 and co-expressed with an activator. Outcomes were consistent with the prediction that readout would be low only with sufficient reconstitution of the inhibitor (FIG. 1E). These initial test cases demonstrate that model-guided design can identify effective topologies, as well as the precise relationship between input component levels and circuit output.

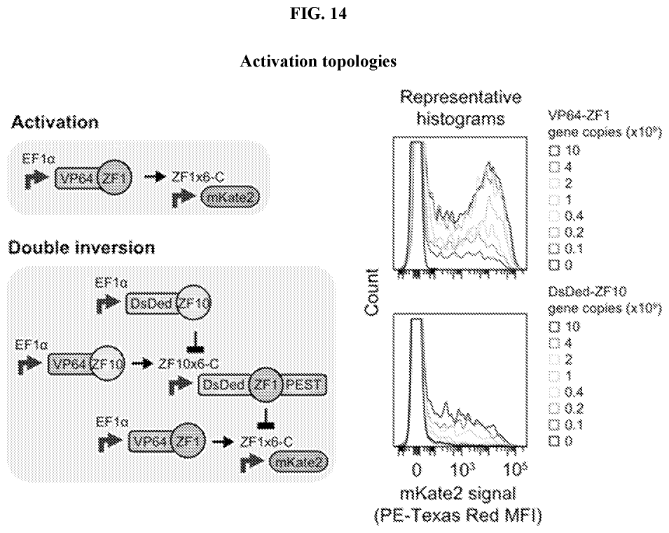

A versatile design framework would enable one to achieve a given performance objective via multiple circuits. Thus, the combined properties of COMET and splicing-based extensions were developed here to provide a sufficient basis for this capability. To investigate, four designs for a NIMPLY gate were compared, each of which utilizes a different mechanism (i.e., topology and/or choice of parts). The first two designs used inhibition mediated by ZF1 (FIG. 1F) or DsDed-ZF1 (FIG. 1G). The third design used splicing of a VP64-intC-ZF1 activator to a DsDed-ZF1 inhibitor, such that the readout would be high only with VP64-intC-ZF1 in sufficient excess of its splicing partner DsDed-intN (FIG. 1H). The fourth design used a double inversion cascade, in which an upstream inhibitor prevented a downstream inhibitor from acting on the reporter (FIG. 1I); this scenario represents a variation on a topology that was previously examined in bacteria (33) and later in mammalian cells with dCas9-TFs (34). All four designs produced NIMPLY as predicted. Next, it was investigated whether splicing could be combined with a cascade, and indeed it was possible to build an AND gate by splitting the cascade's upstream inhibitor into DsDed-intN and intC-ZF10 (FIG. 1J). Unlike standard ZFa-mediated activation, this activation via double inversion exhibited ultrasensitivity (Hill coefficient n=2.8)—a signal transformation in which a small change in input yielded a large change in output, and high output was produced only with sufficient input (FIG. 1K, FIG. 14). Ultrasensitivity buffered the circuit against low inputs, such that the output remained low for input levels that in the standard activation case would have produced half-maximal activation.

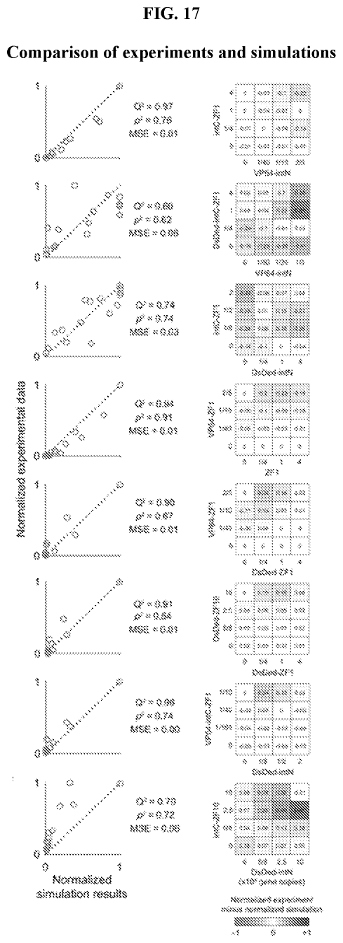

Across the panel, five of the eight gates exhibited a goodness of prediction metric (comparing all simulated and observed outcomes, Q²) of at least 90%, indicating a high capacity for predicting dose response landscapes that had not been used in model training (FIG. 17). Even for the gate with the lowest Q² (IMPLY, FIG. 1D, FIG. 17), the model correctly predicted the trend across most input dose combinations. Altogether, these results demonstrate the feasibility of model-guided design of logic gates in mammalian cells, and that the choice of parts and mechanism yields predictable performance characteristics.

### Example 4—Compression of Circuit Design Using Functional Modularity

A putative advantage of orthogonal parts like COMET TFs and promoters is that these parts may be used together without disrupting their functions. However, simply appending modules can lead to inefficient and cumbersome designs, and thus, one focus of the current approach was achieving genetic compactness as well as performance. Enhancing compactness could eliminate potential failure modes and reduce cargo size for gene delivery vehicles. Genetic compression—reducing the number of components for a given specification—has been investigated by using recombinase-mediated DNA rearrangement (35) and by borrowing from a software engineering strategy to eliminate redundancy (36). Here, it was sought to implement a previously unexplored form of topological compaction based on protein multi-tasking (FIG. 2A). Because the disclosed genetic parts operate through direct interactions without relying on long-range mechanisms such as chromatin modification, they may exhibit functional modularity, i.e., domains could be concatenated and retain their functions. This property would be of great utility by enabling the use of multi-tasking proteins to act at multiple promoters or in both transcriptional and post-translational roles, to execute multiple functions in an efficient fashion.

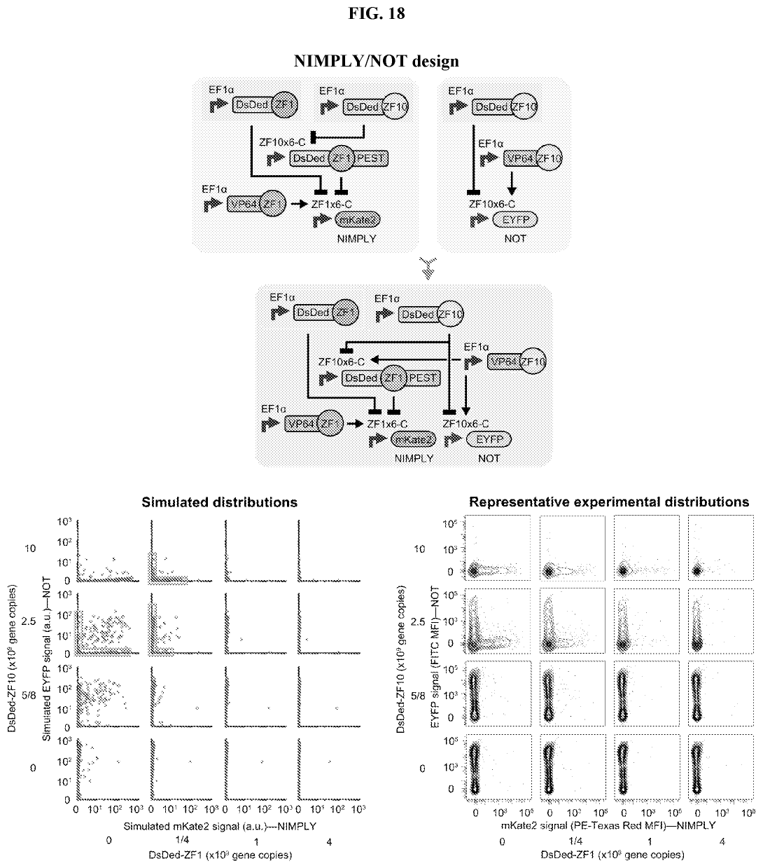

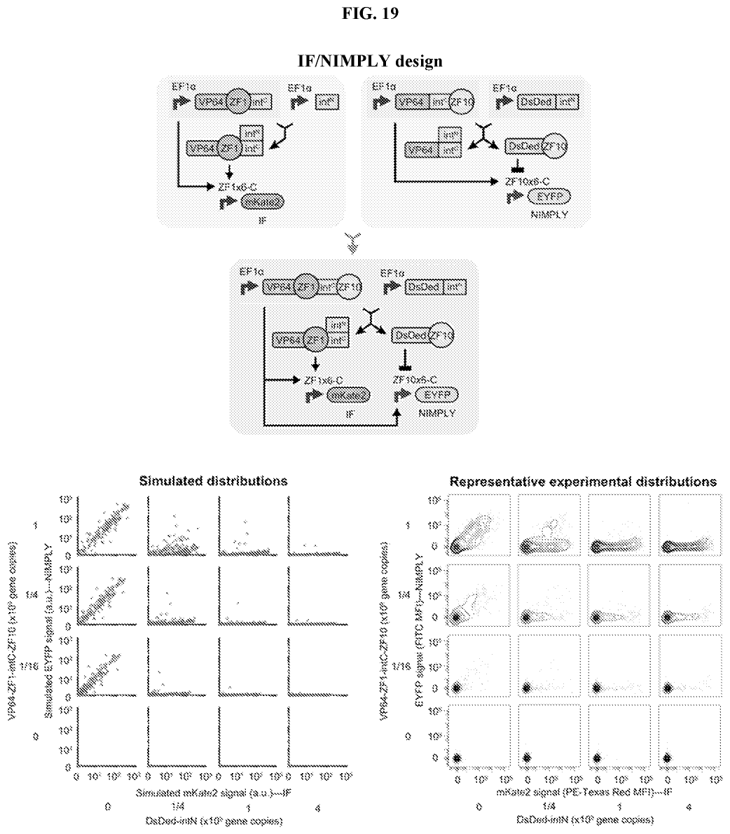

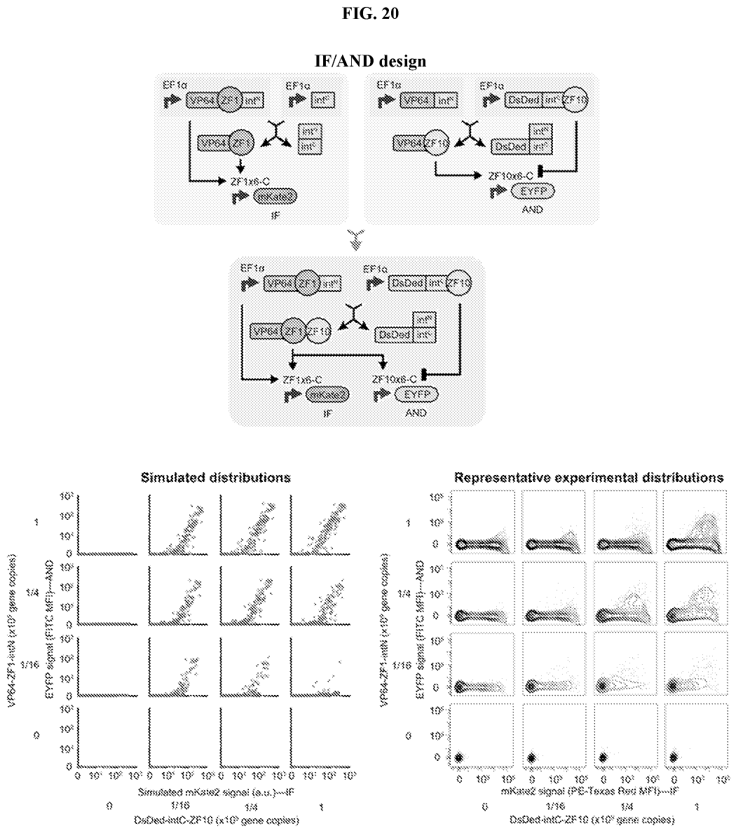

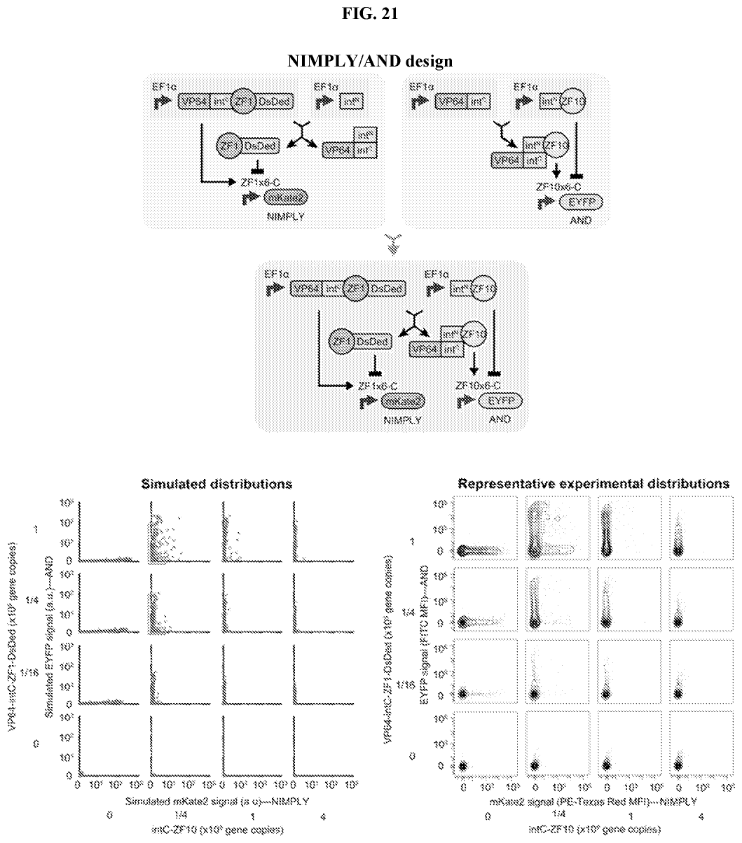

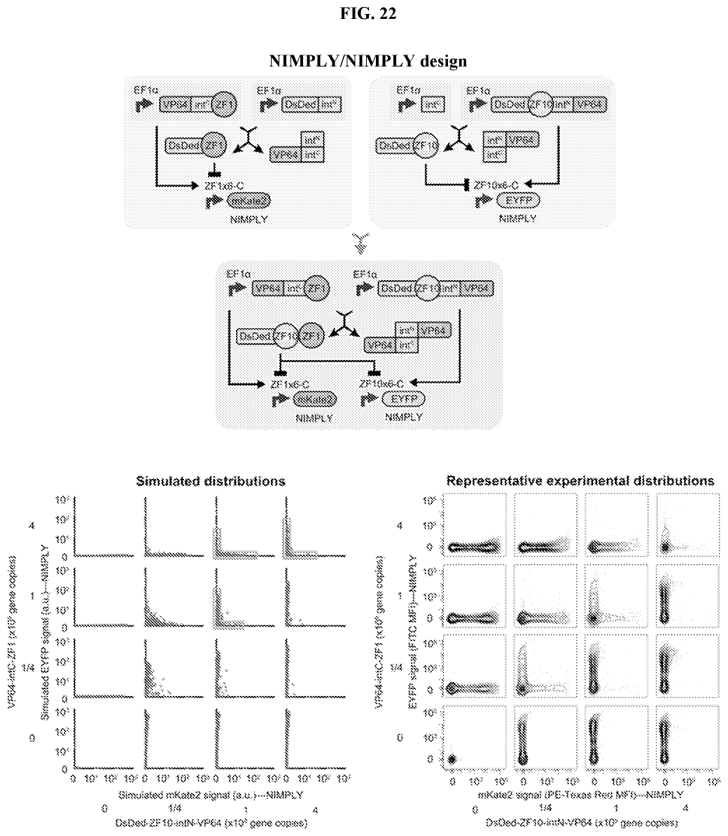

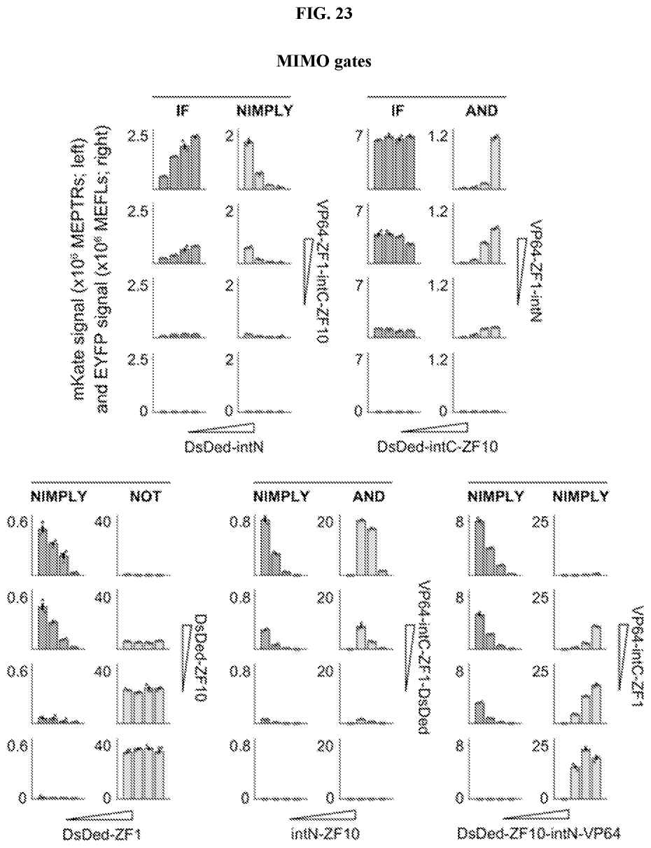

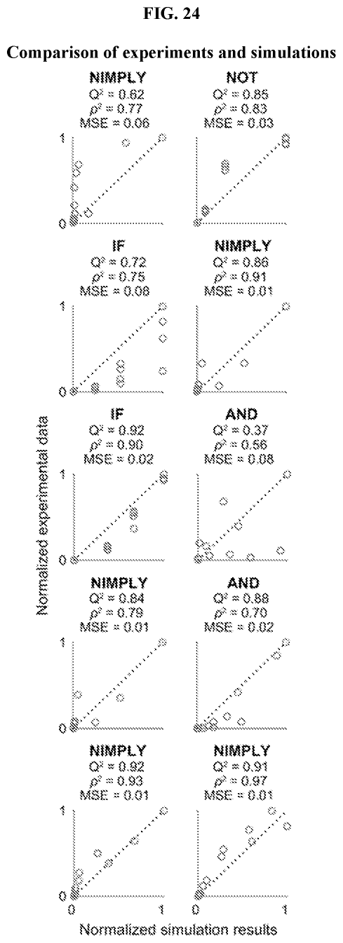

It was investigated whether functional modularity could enable the design of compact multi-input multi-output (MIMO) systems. Ultimately, this capability could support the encoding of sophisticated decision-making strategies in which cells take different actions in different situations. As a base case, a NIMPLY gate and a NOT gate were appended in a non-compact manner, and the combination functioned as expected (FIG. 2B, FIG. 18). This success demonstrated the potential for composite functions, but it brings no efficiency relative to the individual gates. To test topological compaction, first, an IF/NIMPLY gate was proposed in which VP64-ZF1-intC-ZF10 would act as a bispecific activator (on two promoters) and interact with an inert DsDed-intN to produce a VP64-ZF1-intC/intN activator and a DsDed-ZF10 inhibitor (FIG. 2C, FIG. 19). The second gate, IF/AND, used an activator and an inhibitor to produce a bispecific activator and an inert protein, through essentially the inverse mechanism of that in the IF/NIMPLY gate (FIG. 2D, FIG. 20). Third, a NIMPLY/AND gate used a VP64-intC-ZF1-DsDed activator and an intN-ZF10 inhibitor to invert their respective activities (FIG. 2E, FIG. 21). The former protein may act as an activator, in that DsDed may not preclude VP64 from conferring activation. Lastly, a NIMPLY/NIMPLY gate used two activators to produce a bifunctional inhibitor and an inert protein (FIG. 2F, FIG. 22). If this circuit had used the same readout for both reporters it would be a XOR gate. Overall, the model predictions explained most of the variance in experimental outcomes, and several cases were in close agreement (≥90% Q²) (FIGS. 23-24). Minor deviations are potentially attributable to effects such as differences in stability for different proteins; however, such effects were not incorporated into the model because increasing model complexity could lead to overfitting. Moreover, the choice to simplify the description of protein stability did not preclude model-guided identification of high-performing designs.

Notably, when performance at the single-cell level was examined, some population-level outcomes were driven by subpopulations of cells. In some circuits, subpopulations induced one reporter or the other, but not both, and thus population outcomes were driven by shifts in subpopulation frequencies (FIGS. 18, 21, 22). In other circuits, this task distribution was not apparent (FIGS. 19, 20). Although neither behavior was an explicitly designed feature, both types of behavior were predicted by simulations. Altogether, the gates described in FIGS. 1 and 2 span a wide range of logical complexity (the number and the layers of implicit gates depicted in the electronic diagrams) and genetic complexity (the number of genes, regulatory connections, and regulatory proteins) (FIG. 2G). The successful development of these circuits without the need for additional tuning demonstrates that this framework is well-suited to overcoming complexity-associated barriers with mammalian genetic program design.

### Example 5—Implementation of Analog Signal Processing

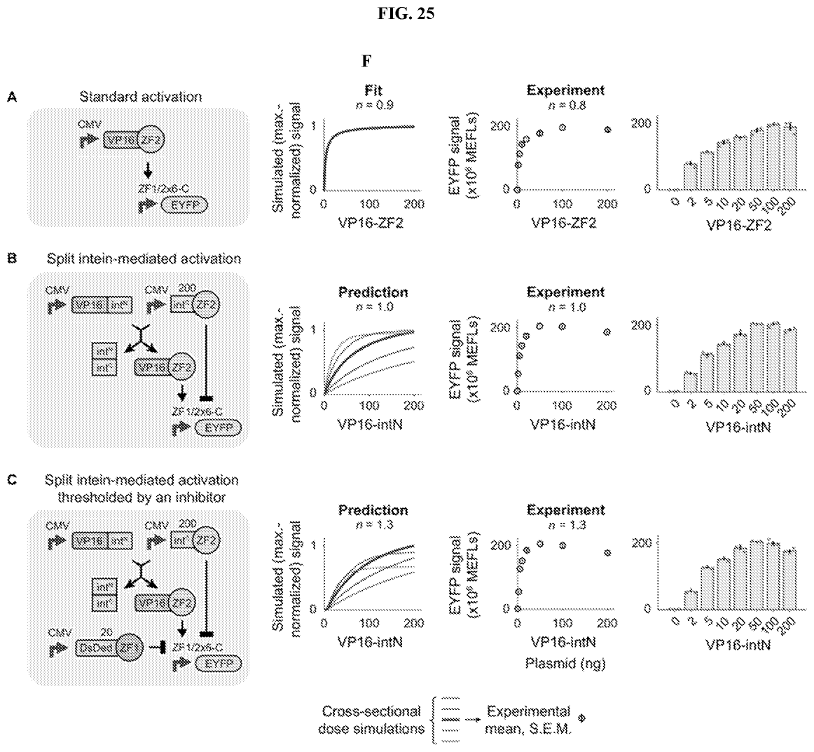

Although digital logic has many uses, biology also processes analog signals for many purposes, and it was next examined whether the disclosed tools could be employed in this way. The first property that for which implementation was sought was ultrasensitivity, which is desirable in engineering sharp activation (37, 38) and is observed in the natural control of processes including cell growth, division, and apoptosis (39). The second property was bandpass concentration filtering, in which an output is produced only when the input falls within a certain range of magnitudes (22, 40). Bandpass concentration filtering is salient for both natural and synthetic spatial patterning (41). To develop a strategy for implementing these properties, mechanistic insights were used. It was determined that ZFa-mediated activation is cooperative at the level of transcription initiation, and in comparing promoter architectures, maximal transcription increased with the number and compactness of binding sites (13). This COMET promoter feature confers high inducibility as well as a high sensitivity to inhibition by proteins that compete for DNA binding. It was also deduced that TF binding to promoter is generally non-cooperative, and transcriptional output from such promoters is not inherently ultrasensitive to ZFa dose (n=1). To construct systems that do exhibit ultrasensitivity (n>1), several strategies were examined in which the output is inhibited only at low activator doses (FIG. 25). The first design made use of the inhibition conferred by intC-ZF1 prior to splicing with a VP16-intN input (FIG. 25B). It was reasoned that at low VP16-intN doses, intC-ZF-mediated inhibition would dominate, and at high doses, transactivation by reconstituted VP16-ZF would dominate. This concept was also tested with the addition of a DsDed-ZF to threshold the response by promoting relatively more inhibition at low input doses (FIG. 25C). However, the increase in ultrasensitivity was modest for these cases, apparently from insufficient inhibition at low activator doses due to decreased protein stability caused by appending the intC domain to the inhibitory ZF (FIG. 9).

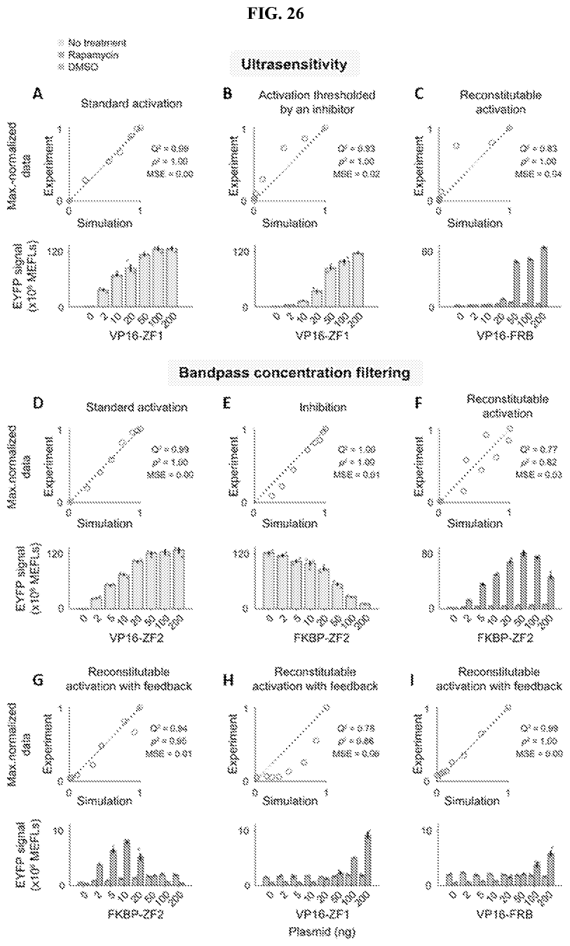

Compared to a ZFa base case (n=1.0) (FIGS. 3A, 26), however, DsDed-ZF thresholding of ZFa-mediated activation did lead to an increase in the Hill coefficient (n=1.9) (FIG. 3B, FIG. 26). This outcome led to consideration of a vehicular analogy: the circuits with DsDed-ZF are akin to applying the brake (inhibition) while applying the accelerator (activation), but a more effective approach would be to release the brake as the accelerator is applied. To realize this concept and circumvent choices that modulate protein stability, a chemically responsive COMET TF (RaZFa) was used in which rapamycin-induced heterodimerization domains FRB and FKBP are fused to an AD and a ZF, respectively. In the presence of rapamycin (which in this scenario is not an input, but rather an environmental species), heterodimerization of VP16-FRB and FKBP-ZF converts FKBP-ZF (brake) into RaZFa (accelerator), which induces the reporter. With rapamycin, the response of this circuit to VP16-FRB input indeed exhibited greater ultrasensitivity (n=3.3), consistent with the prediction (FIG. 3C, FIG. 26). Thus, in this system, ultrasensitivity can arise through cascades (FIG. 1) or reconstitution (FIG. 3), and neither mechanism requires the cooperativity in TF-DNA binding that is often associated with ultrasensitive responses.

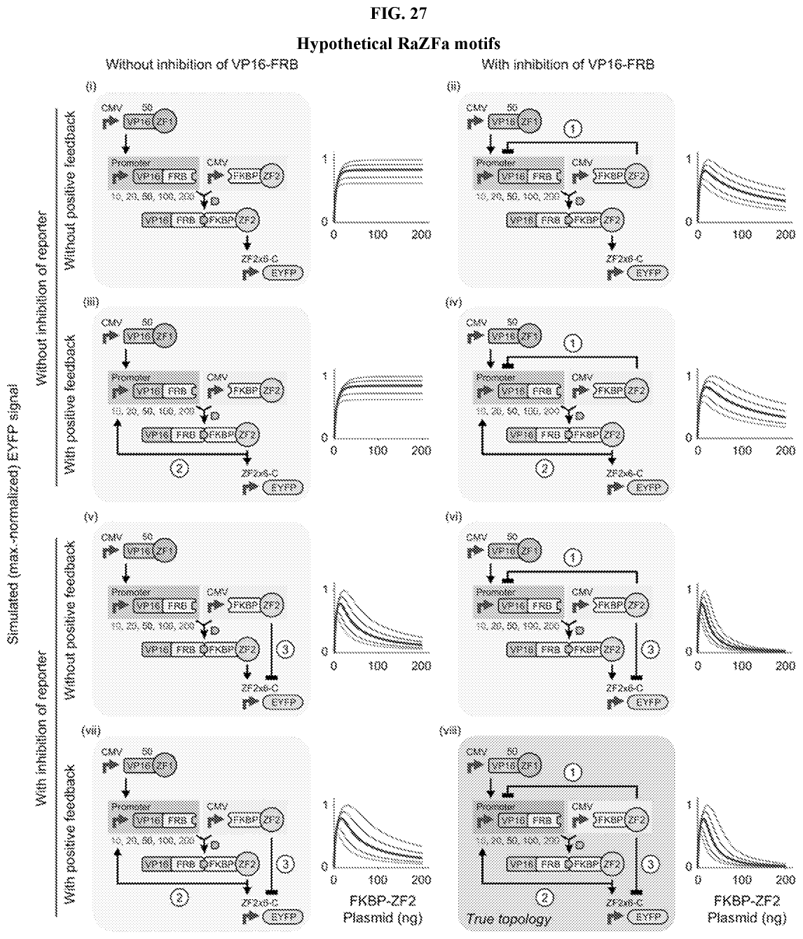

Next, circuits to implement bandpass concentration filtering were investigated. The strategy was to use mechanisms that inhibit reporter output only at high doses of activator input, and the predictions were based on a fitted ZFa base case (FIG. 3D, FIG. 26). Although FKBP-ZF is necessary for RaZFa-mediated activation, it was expected that excess FKBP-ZF would be inhibitory. It was confirmed that FKBP-ZF acted as an inhibitor (FIG. 3E, FIG. 26), and an RaZFa test circuit was implemented; the response to FKBP-ZF input showed a peak in output, but no sharp upper threshold, as predicted (FIG. 3F, FIG. 26). Based on these results, a new topology was designed to achieve a sharper bandpass. Of the regulation within this design, the two paths of negative regulation from FKBP-ZF (and not the positive feedback from RaZFa) appeared to be most important for sharpening the bandpass (FIG. 27). For the primary input to the bandpass, FKBP-ZF, it was expected that at zero dose, there would be no activation; at moderate doses, there would be activation; and at high doses, excess FKBP-ZF would both decrease reconstitution (by inhibiting induction of VP16-FRB) and inhibit the reporter. The experimental outcomes closely matched the prediction of a bandpass with a sharp upper threshold (FIG. 3G, FIG. 26). Furthermore, when VP16-ZF or VP16-FRB doses were varied, the responses were activating as predicted (FIG. 3H-I, FIG. 26), demonstrating a predictive capacity across multiple inputs for the system. These results demonstrate that the disclosed parts and approach are suitable for designing analog behaviors, as well as digital logic gates.

### Example 6—Integration of Genetic Circuits with Sensors to Build Sense-and-Respond Functions

While the predictive design of genetic programs is a substantial technical advance in and of itself, employing this capability to enable many potential applications will require integrating genetic circuits with native or synthetic parts that sense and modulate the state of the cell or its environment. A recurring challenge associated with this goal is level-matching the output of a sensor to the input requirements of a downstream circuit (32, 42). It was investigated whether the disclosed designed circuits could overcome this challenge and be effectively linked to sensors without requiring laborious trial-and-error tuning. Simulations suggested that adding an upstream layer of signal processing (i.e., for sensing) is feasible, since in the model, ZFa can be arranged in series without prohibitively driving up background or dampening induced signal (FIG. 28).

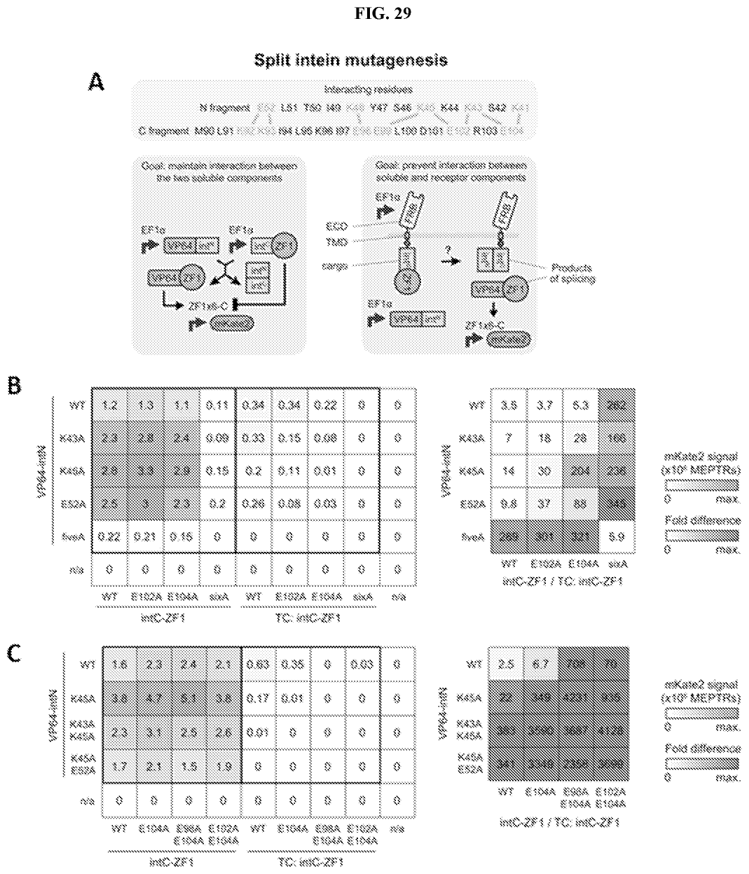

Two classes of synthetic sensors (intracellular and transmembrane) were considered for which it was hypothesized that signaling (i.e., sensor output) could be coupled to COMET-based circuits. For the intracellular sensor, a new TF—ABA-ZFa was built, which was analogous to RaZFa—by fusing the abscisic acid (ABA)-binding domains PYL1 and ABI1 (43) to an AD and a ZF, respectively. For transmembrane sensing, the modular extracellular sensor architecture (MESA)—a self-contained receptor and signal transduction system that transduces ligand binding into orthogonal regulation of target genes (44, 45)—was selected. In this mechanism, ligand-mediated dimerization of two transmembrane proteins called the target chain (TC) and protease chain (PC) promoted PC-mediated proteolytic trans-cleavage of a TC-bound TF. Several strategies were explored for building COMET-compatible MESA based on a recently reported improved MESA design (46) and the parts developed in the current study (FIGS. 29-32). The best performance was observed using rapalog-inducible COMET-MESA that release either ZFa for activating signaling or DsDed-ZF for inhibitory signaling (the latter represents a new function for MESA receptors) (FIG. 32); the ZFa-releasing COMET-MESA receptor was carried forward. Both sensors displayed excellent performance in terms of reporter induction upon ligand treatment (FIG. 4A,B). For ABA-ZF2a (ZF2a was selected for its potency stemming from cooperative transcriptional activation (13)), ligand-independent signal was unobservable, and induced signal was high, yielding perfect performance (FIG. 4A). For Rapa-MESA-ZF6a (ZF6a was also selected for its potency), the ligand-inducible fold difference in signal was˜200×(FIG. 4B), which is several fold higher than was observed for recently reported receptors based on tTA (46), and also higher than the fold difference observed for a high-performing MESA that employs a distinct mechanism (47). Thus, Rapa-MESA-ZF6a is the highest performing MESA reported to date. Both sensors have a low off state and a high on state, apparently benefitting from the advantageous property of COMET promoter-based cooperativity.

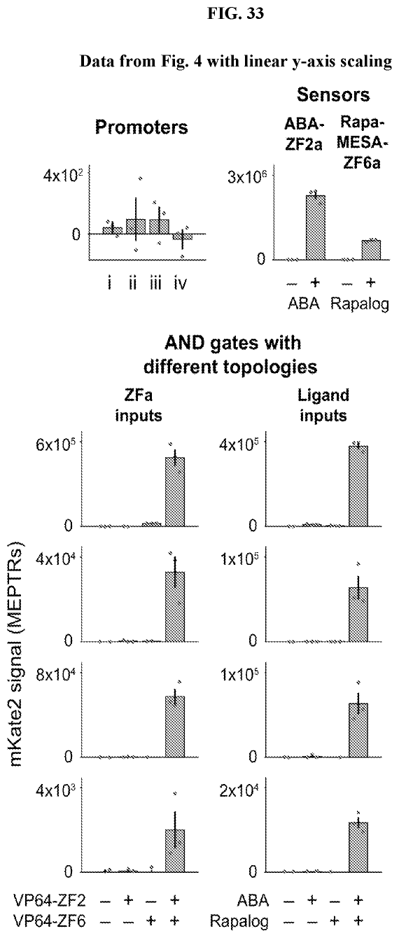

The two validated sensors were carried forward and downstream circuits comprising genetic parts and designed topologies from this disclosure were examined to determine whether they could be seamlessly linked with the new input layer. To this end, a panel of four synonymous topologies were designed that implement AND logic through different mechanisms (FIG. 4C): 1) a hybrid promoter with alternating TF sites (based on a similar architecture from the original COMET study (13)), 2) splicing (as in FIG. 1C), 3) splicing with DsDed (as in FIGS. 1D, 2D for tighter inhibition), and 4) and splicing with feedback (as in FIG. 3G-I). All four topologies exhibited AND behavior when tested using ZFa as inputs (FIG. 4D), demonstrating the versatility for attaining a given objective in multiple ways. Moreover, when coupled to ligand-activated sensors, these circuits still conferred AND behavior, and performance was maintained (i.e., fold induction with two ligands remained much greater than with each ligand individually) in carrying out this more complex sensing function (FIG. 4E). A comparison across the designs provides some insights. The hybrid promoter in topology 1 was high-performing, and the splicing topologies in 2-4 generally yielded improvement over 1, despite the additional regulatory layer, by reducing the output generated from either single input alone to near reporter-only background (FIG. 33, shown with linear scaling). Of the topologies examined, 2 and 3 were the most effective at producing a high output when both inputs were present and low output when either input was present alone. These results demonstrate that genetic programs can be designed by a predictive model-driven process, and then these programs can be readily linked to different classes of sensors to implement high-performing sensing and processing functions.

All patents and publications mentioned in the specification are indicative of the levels of those of ordinary skill in the art to which the disclosure pertains. All patents and publications are herein incorporated by reference to the same extent as if each individual publication was specifically and individually indicated to be incorporated by reference.

Further, one skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting embodiments of the disclosure.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

References Cited
1. P. Saxena et al., A programmable synthetic lineage-control network that differentiates human IPSCs into glucose-sensitive insulin-secreting beta-like cells. Nat Commun 7, 11247 (2016).
2. P. Guye et al., Genetically engineering self-organization of human pluripotent stem cells into a liver bud-like tissue using Gata6. Nat Commun 7, 10243 (2016).
3. K. T. Roybal et al., Precision tumor recognition by T cells with combinatorial antigen-sensing circuits. Cell 164, 770-779 (2016).
4. M. A. Fischbach, J. A. Bluestone, W. A. Lim, Cell-based therapeutics: the next pillar of medicine. Sci Trans Med 5, 179ps177 (2013).
5. T. Kitada, B. DiAndreth, B. Teague, R. Weiss, Programming gene and engineered-cell therapies with synthetic biology. Science 359, eaad1067 (2018).
6. M. Xie, M. Fussenegger, Designing cell function: assembly of synthetic gene circuits for cell biology applications. Nat Rev Mol Cell Biol 19, 507-525 (2018).
7. A. L. Slusarczyk, A. Lin, R. Weiss, Foundations for the design and implementation of synthetic genetic circuits. Nat Rev Genet 13, 406-420 (2012).
8. F. Lienert, J. J. Lohmueller, A. Garg, P. A. Silver, Synthetic biology in mammalian cells: next generation research tools and therapeutics. Nat Rev Mol Cell Biol 15, 95-107 (2014).
9. A. A. K. Nielsen et al., Genetic circuit design automation. Science 352, aac7341 (2016).
10. Y. Chen et al., Genetic circuit design automation for yeast. Nat Microbiol, (2020).
11. C. J. Bashor et al., Complex signal processing in synthetic gene circuits using cooperative regulatory assemblies. Science 364, 593-597 (2019).
12. J. J. Lohmueller, T. Z. Armel, P. A. Silver, A tunable zinc finger-based framework for Boolean logic computation in mammalian cells. Nucleic Acids Res 40, 5180-5187 (2012).
13. P. S. Donahue et al., The COMET toolkit for composing customizable genetic programs in mammalian cells. Nat Commun 11, 779 (2020).
14. F. Lienert et al., Two- and three-input TALE-based AND logic computation in embryonic stem cells. Nucleic Acids Res 41, 9967-9975 (2013).
15. R. Gaber et al., Designable DNA-binding domains enable construction of logic circuits in mammalian cells. Nat Chem Biol 10, 203-208 (2014).
16. T. Lebar, A. Verbič, A. Ljubetič, R. Jerala, Polarized displacement by transcription activator-like effectors for regulatory circuits. Nat Chem Biol 15, 80-87 (2018).
17. Z. Chen et al., De novo design of protein logic gates. Science 368, 78-84 (2020).
18. D. Ma, S. Peng, Z. Xie, Integration and exchange of split dCas9 domains for transcriptional controls in mammalian cells. Nat Commun 7, 13056 (2016).
19. H. Kim, D. Bojar, M. Fussenegger, A CRISPR/Cas9-based central processing unit to program complex logic computation in human cells. Proc Natl Acad Sci USA 116, 7214-7219 (2019).
20. B. Angelici, E. Mailand, B. Haefliger, Y. Benenson, Synthetic biology platform for sensing and integrating endogenous transcriptional inputs in mammalian cells. Cell Rep 16, 2525-2537 (2016).
21. S. Ausländer, D. Ausländer, M. Müller, M. Wieland, M. Fussenegger, Programmable single-cell mammalian biocomputers. Nature 487, 123-127 (2012).
22. X. J. Gao, L. S. Chong, M. S. Kim, M. B. Elowitz, Programmable protein circuits in living cells. Science 361, 1252-1258 (2018).
23. T. Fink et al., Design of fast proteolysis-based signaling and logic circuits in mammalian cells. Nat Chem Biol 15, 115-122 (2018).
24. Z. Kis, H. S. Pereira, T. Homma, R. M. Pedrigi, R. Krams, Mammalian synthetic biology: emerging medical applications. J R Soc Interface 12, 20141000 (2015).
25. N. Davidsohn et al., Accurate predictions of genetic circuit behavior from part characterization and modular composition. ACS Synth Biol 4, 673-681 (2014).
26. C. Briat, A. Gupta, M. Khammash, Antithetic integral feedback ensures robust perfect adaptation in noisy biomolecular networks. Cell Syst 2, 15-26 (2016).
27. D. Del Vecchio, H. Abdallah, Y. Qian, J. J. Collins, A blueprint for a synthetic genetic feedback controller to reprogram cell fate. Cell Syst 4, 109-120 (2017).
28. G. Lillacci, Y. Benenson, M. Khammash, Synthetic control systems for high performance gene expression in mammalian cells. Nucleic Acids Res 46, 9855-9863 (2018).
29. M. Vila-Perelló, T. W. Muir, Biological applications of protein splicing. Cell 143,191-200 (2010).
30. P. Carvajal-Vallejos, R. Pallissé, H. D. Mootz, S. R. Schmidt, Unprecedented rates and efficiencies revealed for new natural split inteins from metagenomic sources. J Biol Chem 287, 28686-28696 (2012).
31. G. S. Baird, D. A. Zacharias, R. Y. Tsien, Biochemistry, mutagenesis, and oligomerization of DsRed, a red fluorescent protein from coral. Proc Natl Acad Sci USA 97, 11984-11989 (2000).
32. R. M. Hartfield, K. A. Schwarz, J. J. Muldoon, N. Bagheri, J. N. Leonard, Multiplexing engineered receptors for multiparametric evaluation of environmental ligands. ACS Synth Biol 6, 2042-2055 (2017).
33. S. Hooshangi, S. Thiberge, R. Weiss, Ultrasensitivity and noise propagation in a synthetic transcriptional cascade. Proc Natl Acad Sci USA 102, 3581-3586 (2005).
34. S. Kiani et al., CRISPR transcriptional repression devices and layered circuits in mammalian cells. Nat Methods 11, 723-726 (2014).
35. N. Lapique, Y. Benenson, Genetic programs can be compressed and autonomously decompressed in live cells. Nat Nanotech 13, 309-315 (2018).
36. J. Beal, T. Lu, R. Weiss, Automatic compilation from high-level biologically-oriented programming language to genetic regulatory networks. PloS One 6, e22490 (2011).
37. E. C. O'Shaughnessy, S. Palani, J. J. Collins, C. A. Sarkar, Tunable signal processing in synthetic MAP kinase cascades. Cell 144, 119-131 (2011).
38. T. Shopera et al., Robust, tunable genetic memory from protein sequestration combined with positive feedback. Nucleic Acids Res 43, 9086-9094 (2015).
39. Q. Zhang, S. Bhattacharya, M. E. Andersen, Ultrasensitive response motifs: basic amplifiers in molecular signalling networks. Open Biol 3, 130031 (2013).
40. D. Greber, M. Fussenegger, An engineered mammalian band-pass network. Nucleic Acids Res 38, e174 (2010).
41. N. S. Scholes, M. Isalan, A three-step framework for programming pattern formation. Curr Opin Chem Biol 40, 1-7 (2017).
42. Y.-H. Wang, K. Y. Wei, C. D. Smolke, Synthetic biology: advancing the design of diverse genetic systems. Annu Rev Chem Biomol Eng 4, 69-102 (2013).
43. Y. Gao et al., Complex transcriptional modulation with orthogonal and inducible dCas9 regulators. Nat Methods 13, 1043-1049 (2016).
44. N. M. Daringer, R. M. Dudek, K. A. Schwarz, J. N. Leonard, Modular extracellular sensor architecture for engineering mammalian cell-based devices. ACS Synth Biol 3, 892-902 (2014).
45. K. A. Schwarz, N. M. Daringer, T. B. Dolberg, J. N. Leonard, Rewiring human cellular input-output using modular extracellular sensors. Nat Chem Biol 13, 202-209 (2017).
46. H. I. Edelstein et al., Elucidation and refinement of synthetic receptor mechanisms. Synth Biol, (In press).
47. T. B. Dolberg et al., Computation-guided optimization of split protein systems. bioRxiv, (2019).
48. Y. Hart, U. Alon, The utility of paradoxical components in biological circuits. Mol Cell 49, 213-221 (2013).
49. A. V. Anzalone et al., Search-and-replace genome editing without double-strand breaks or donor DNA. Nature 576, 149-157 (2019).
50. J. J. Muldoon. (Zenodo, https://zenodo.org/record/4013993, 2020).
51. B. Dassa, N. London, B. L. Stoddard, O. Schueler-Furman, S. Pietrokovski, Fractured genes: a novel genomic arrangement involving new split inteins and a new homing endonuclease family. Nucleic Acids Res 37, 2560-2573 (2009).
52. P. Carvajal-Vallejos, R. Pallissé, H. D. Mootz, S. R. Schmidt, Unprecedented rates and efficiencies revealed for new natural split inteins from metagenomic sources. J Biol Chem 287, 28686-28696 (2012).
53. M. Hermann et al., Binary recombinase systems for high-resolution conditional mutagenesis. Nucleic Acids Res 42, 3894-3907 (2014).
54. D. Pan et al., An intein-mediated modulation of protein stability system and its application to study human cytomegalovirus essential gene function. Sci Rep 6, 26167 (2016).
55. J. A. Gramespacher, A. J. Stevens, D. P. Nguyen, J. W. Chin, T. W. Muir, Intein zymogens: conditional assembly and splicing of split inteins via targeted proteolysis. J Am Chem Soc 139, 8074-8077 (2017).
56. J. K. Böcker, W. Dorner, H. D. Mootz, Light-control of the ultra-fast Gp41-1 split intein with preserved stability of a genetically encoded photo-caged amino acid in bacterial cells. Chem Commun 55, 1287-1290 (2019).
57. A.-L. Bachmann, H. D. Mootz, An unprecedented combination of serine and cysteine nucleophiles in a split intein with an atypical split site. J Biol Chem 290, 28792-28804 (2015).
58. H. M. Beyer, K. M. Mikula, M. Li, A. Wlodawer, H. Iwaï, The crystal structure of the naturally split gp41-1 intein guides the engineering of orthogonal split inteins from cis-splicing inteins. FEBS J287, 1886-1898 (2019).
59. N. H. Shah, E. Eryilmaz, D. Cowburn, T. W. Muir, Naturally split inteins assemble through a “capture and collapse” mechanism. J Am Chem Soc 135, 18673-18681 (2013).
60. P. S. Donahue et al., The COMET toolkit for composing customizable genetic programs in mammalian cells. Nat Commun 11, 779 (2020).
61. A. S. Khalil et al., A synthetic biology framework for programming eukaryotic transcription functions. Cell 150, 647-658 (2012).
62. A. Chavez et al., Highly efficient Cas9-mediated transcriptional programming. Nat Methods 12, 326-328 (2015).
63. N. M. Daringer, R. M. Dudek, K. A. Schwarz, J. N. Leonard, Modular extracellular sensor architecture for engineering mammalian cell-based devices. ACS Synth Biol 3, 892-902 (2014).
64. S.-Y. Park et al., Abscisic acid inhibits type 2C protein phosphatases via the PYR/PYL family of START proteins. Science 324, 1068-1071 (2009).
65. Y. Ma et al., Regulators of PP2C phosphatase activity function as abscisic acid sensors. Science 324, 1064-1068 (2009).
66. K. Melcher et al., A gate-latch-lock mechanism for hormone signalling by abscisic acid receptors. Nature 463, 602-608 (2009).
67. K.-i. Miyazono et al., Structural basis of abscisic acid signalling. Nature 462, 609-614 (2009).
68. P. Yin et al., Structural insights into the mechanism of abscisic acid signaling by PYL proteins. Nat Struct Mol Biol 16, 1230-1236 (2009).
69. Y. Gao et al., Complex transcriptional modulation with orthogonal and inducible dCas9 regulators. Nat Methods 13, 1043-1049 (2016).
70. P. Loetscher, G. Pratt, M. Rechsteiner, The C terminus of mouse ornithine decarboxylase confers rapid degradation on dihydrofolate reductase. J Biol Chem 17, 11213-11220 (1991).
71. X. Duportet et al., A platform for rapid prototyping of synthetic gene networks in mammalian cells. Nucleic Acids Res 44, 2677-2690 (2014).
72. L. Bintu et al., Dynamics of epigenetic regulation at the single-cell level. Science 351, 720-724 (2016).
73. R. M. Hartfield, K. A. Schwarz, J. J. Muldoon, N. Bagheri, J. N. Leonard, Multiplexing engineered receptors for multiparametric evaluation of environmental ligands. ACS Synth Biol 6, 2042-2055 (2017).
74. G. S. Baird, D. A. Zacharias, R. Y. Tsien, Biochemistry, mutagenesis, and oligomerization of DsRed, a red fluorescent protein from coral. Proc Natl Acad Sci USA 97, 11984-11989 (2000).

1. An engineered genetic circuit comprising:
(a) one or more engineered proteins selected from the group consisting of:
an engineered protein that activates gene expression, wherein the engineered protein comprises a DNA binding domain, a transcription activator domain, and at least one split intein on the C-terminus or N-terminus of the DNA binding domain and/or the transcription activator domain;
(ii) an engineered protein that inhibits gene expression, the engineered protein comprising a DNA binding domain, a transcription inhibitor domain, and at least one split intein on the C-terminus or N-terminus of the DNA binding domain and/or the transcription inhibitor domain; and
(iii) a combination of two engineered proteins comprising a first engineered protein comprising a DNA binding domain fused to a dimerization domain, and a second engineered protein comprising a transcription regulator domain fused to a dimerization domain, wherein the dimerization domains of the two engineered proteins dimerize in the presence of a stimulus to which the dimerization domains of the two engineered proteins bind, and wherein and the first engineered protein and the second engineered protein each comprise at least one split intein; and
(b) one or more engineered expression vectors comprising a minimal promoter and one or more DNA binding sites for the DNA binding domains of the engineered proteins of (a), and optionally a gene of interest that is expressed from the minimal promoter.

2. The engineered genetic circuit of claim 1 comprising the engineered protein of (i) and the engineered protein of (ii).

3. The engineered genetic circuit of claim 1, wherein the DNA binding domain of the one or more engineered proteins of (i), (ii), and (iii) comprises one or more zinc fingers.

4. The engineered genetic circuit of claim 1, wherein the DNA binding domain of the one or more engineered proteins of (i), (ii), and (iii) comprises 2, 3, or more zinc fingers.

5. The engineered genetic circuit of claim 1, wherein the engineered proteins are fusion proteins comprising heterologous domains.

6. The engineered genetic circuit of claim 1, wherein the transcription activator domain of the engineered protein of (i), (ii), and/or (iii) comprises a domain from a transcription activator selected from the group consisting of Herpes simplex virus protein 16 (VP16), a synthetic tetramer of VP16 (VP64), nuclear factor (NF) kappa-B (p65), heat shock transcription factor 1 (HSF1), replication and transcription activator (RTA) of the gamma-herpesvirus family, p53, an acidic domain (also known as “acid blobs” or “negative noodles,” rich in D and E amino acids, present in Ga14, Gcn4 and VP16), a glutamine-rich domain (which may comprise multiple repetitions like “QQQXXXQQQ (SEQ ID NO: 4),” like those present in transcription factor Sp1), a proline-rich domains (which may comprise repetitions like “PPPXXXPPP (SEQ ID NO: 5,” like those present in c-jun, AP2, and October 2), an isoleucine-rich domain (which may comprise repetitions of “IIXXII (SEQ ID NO: 6),” like those present in NTF-1), and a multipartite activator.

7. The engineered genetic circuit of claim 1, wherein the engineered protein of (ii) inhibits activation of transcription by the engineered protein of (i).

8. The engineered genetic circuit of claim 1, wherein the transcription regulator domain of the second engineered protein of the combination of engineered proteins of (iii) is a transcription activator domain optionally selected from the group consisting of Herpes simplex virus protein 16 (VP16), a synthetic tetramer of VP16 (VP64), nuclear factor (NF) kappa-B (p65), heat shock transcription factor 1 (HSF1), replication and transcription activator (RTA) of the gamma-herpesvirus family, p53, an acidic domain (also known as “acid blobs” or “negative noodles,” rich in D and E amino acids, present in Ga14, Gcn4 and VP16), a glutamine-rich domain (which may comprise multiple repetitions like “QQQXXXQQQ (SEQ ID NO: 4),” like those present in transcription factor Sp1), a proline-rich domains (which may comprise repetitions like “PPPXXXPPP (SEQ ID NO: 5),” like those present in c-jun, AP2, and October 2), an isoleucine-rich domain (which may comprise repetitions of “IIXXII (SEQ ID NO:6),” like those present in NTF-1), and a multipartite activator.

9. The engineered genetic circuit of claim 1, wherein the engineered proteins of (i) or (ii) are present in an exogenous extracellular sensor.

10. The engineered genetic circuit of claim 9, wherein the extracellular sensor comprises: (a) a ligand binding domain, (b) a transmembrane domain, (c) a protease cleavage site, and (d) the engineered protein of (i) or (ii).

11. The engineered genetic circuit of claim 1, wherein the split intein is a wild-type split intein.

12. The engineered genetic circuit of claim 1, wherein the at least one split intein is a mutated split intein.

13. The engineered genetic circuit of claim 1, wherein the at least one split intein is appended to the N-terminus of the engineered protein.

14. The engineered genetic circuit of claim 13, wherein the split intein comprises 1 or an amino acid sequence that possesses at least about 70%, at least about 75%, at least about 80%, at least about 85%, at least about 90%, at least about 95%, at least about 96%, at least about 97%, at least about 98%, or at least about 99% identity thereto.

15. The engineered genetic circuit of claim 1, wherein the at least one split intein is appended to the C-terminus of the engineered protein.

16. The engineered genetic circuit of claim 15, wherein the split intein comprises 3 or an amino acid sequence that possesses at least about 70%, at least about 75%, at least about 80%, at least about 85%, at least about 90%, at least about 95%, at least about 96%, at least about 97%, at least about 98%, or at least about 99% identity thereto.

17. The engineered genetic circuit of claim 1, wherein the circuit components are eukaryotic.

18. The engineered genetic circuit of claim 1, wherein the circuit components are mammalian.

19. The engineered genetic circuit of claim 1, wherein the stimulus is a ligand, exposure to light, removal from light, phosphorylation, dephosphorylation, a post-translational modification of the dimerization domain, a change in the state of the environment in which the engineered genetic circuit is expressed.

20. An engineered genetic circuit, comprising:
(a) a first engineered protein that activates gene expression, the first engineered protein comprising a first DNA binding domain, a first transcription activator domain, and at least one split intein on the C-terminus or N-terminus of the first DNA binding domain and/or the first transcription activator domain;
(b) a first engineered expression vector comprising a minimal promoter and first DNA binding sites for the first DNA binding domain of the first engineered protein, and a first gene of interest that is expressed from the minimal promoter, wherein the gene of interest encodes a second engineered protein, the second engineered protein comprising a second DNA binding domain, a second transcription activator domain, and at least one split intein on the C-terminus or N-terminus of the second DNA binding domain and/or the second transcription activator domain; and
(c) a second engineered expression vector comprising a minimal promoter and second DNA binding sites for the second DNA binding domain of the second engineered protein, and a second gene of interest that is expressed from the minimal promoter, wherein the second gene of interest encodes a detectable reporter protein;
wherein the first engineered protein increases expression from the first engineered expression vector and the second engineered protein increases expression from the second engineered vector.

21. An exogenous extracellular sensor system comprising:
a first exogenous extracellular sensor component comprising:
(a) a ligand binding domain,
(b) a transmembrane domain,
(c) a protease cleavage site, and
(d) an engineered protein domain comprising a DNA binding domain, a transcription activator domain, and at least one split intein on the C-terminus or N-terminus of the DNA binding domain and/or the transcription activator domain;
(ii) a second exogenous extracellular sensor component comprising
(a) a ligand binding domain,
(b) a transmembrane domain, and
(c) a protease domain; and, optionally,
(iii) an engineered expression vector comprising a minimal promoter and one or more DNA binding sites for the DNA binding domains of the first exogenous extracellular sensor, and, optionally, a gene of interest that is expressed from the minimal promoter;
wherein the ligand binding domain of the first exogenous extracellular sensor component and the ligand binding domain of the second exogenous extracellular sensor component bind to the same ligand to form a tertiary complex;
wherein the protease domain of the second exogenous extracellular sensor component cleaves the protease cleavage site of the first exogenous extracellular sensor component to release the engineered protein domain comprising the DNA binding domain and transcription activator domain;
and wherein the DNA binding domain of the engineered protein domain binds to the one or more DNA binding sites of the engineered expression vector and increases expression from the minimal promoter of the engineered expression vector.

22. A host cell comprising the engineered genetic circuit of claim 1.

23. The host cell of claim 22, wherein the cell is eukaryotic.

24. The host cell of claim 22, wherein the cell is mammalian.


SEQUENCE LISTING (8 sequences)

SEQ ID NO: 1 - protein, 88 aa, Artificial Sequence
  source: /note=“Description of Artificial Sequence Synthetic polypeptide”
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 2 - protein, 5 aa, Artificial Sequence
  source: /note=“Description of Artificial Sequence Synthetic peptide”
TRSGY

SEQ ID NO: 3 - protein, 38 aa, Artificial Sequence
  source: /note=“Description of Artificial Sequence Synthetic polypeptide”
MMLKKILKIEELDERELIDIEVSGNHLFYANDILTHNS

SEQ ID NO: 4 - protein, 9 aa, Artificial Sequence
  source: /note=“Description of Artificial Sequence Synthetic peptide”
  MOD_RES (4)..(6): Any amino acid
QQQXXXQQQ

SEQ ID NO: 5 - protein, 9 aa, Artificial Sequence
  source: /note=“Description of Artificial Sequence Synthetic peptide”
  MOD_RES (4)..(6): Any amino acid
PPPXXXPPP

SEQ ID NO: 6 - protein, 6 aa, Artificial Sequence
  source: /note=“Description of Artificial Sequence Synthetic peptide”
  MOD_RES (3)..(4): Any amino acid
IIXXII

SEQ ID NO: 7 - protein, 12 aa, Artificial Sequence
  source: /note=“Description of Artificial Sequence Synthetic peptide”
KSKKKSYKITLE

SEQ ID NO: 8 - protein, 15 aa, Artificial Sequence
  source: /note=“Description of Artificial Sequence Synthetic peptide”
MLKKILKIEELDERE